(19) United States
(12) Patent Application Publication
(10) Pub. No.: US 2026/0345751 A1
(43) Pub. Date: Nov. 5, 2026

(54) COMPOSITION FOR PREVENTING AGING COMPRISING PLASMA-DERIVED PROTEIN

(71) Applicant: DONGGUK UNIVERSITY INDUSTRY-ACADEMIC COOPERATION FOUNDATION, Seoul (KR)

(72) Inventors: Jongpil KIM, Seoul (KR); Junyeop KIM, Guri-si (KR); Yerim HWANG, Ansan-si (KR)

(73) Assignee: DONGGUK UNIVERSITY INDUSTRY-ACADEMIC COOPERATION FOUNDATION, Seoul (KR)

(21) Appl. No.: 19/121,295

(22) PCT Filed: Oct. 18, 2023
(86) PCT No.: PCT/KR2023/016116

§ 371 Date: Jun. 11, 2025

(30) Foreign Application Priority Data
Oct. 18, 2022 (KR) 10-2022-0134415
May 12, 2023 (KR) 10-2023-0061859

(51) Int. Cl. A61K 38/53; A61K 38/17; A61K 38/51; A61K 38/57; A61K 48/00; A61P 1/16; A61P 3/00; A61P 9/10; A61P 19/00; A61P 25/16; A61P 25/28; A61P 43/00

(52) U.S. Cl. CPC A61K 38/53; A61K 38/1709; A61K 38/51; A61K 38/57; A61K 48/0033; A61K 48/005; A61P 1/16; A61P 3/00; A61P 9/10; A61P 19/00; A61P 25/16; A61P 25/28; A61P 43/00; C12Y 402/01011; C12Y 603/02002

(57) ABSTRACT

The present disclosure relates to an anti-aging composition comprising GCLC, ENO1, Map1s, and KNG1 derived from plasma treated with Oct4, Sox2, Klf4 and c-Myc and various combinations thereof, and use thereof.

The various combinations of GCLC, ENO1, Map1s, and KNG1 of the present disclosure improve age-related phenotypes in vitro and in vivo, and improve a disease induced by aging of vascular endothelial cells, a disease induced by aging of organs, a disease induced by aging of nerves, a degenerative brain disease, etc., which are caused by aging, and thus can prevent, improve or treat an age-related disease.

### TECHNICAL FIELD

The present disclosure relates to an anti-aging composition including GCLC, ENO1, Map1s, and KNG1 derived from plasma treated with Oct4, Sox2, Klf4 and c-Myc and various combinations thereof, and use thereof.

### BACKGROUND ART

Terminally differentiated somatic cells can be reprogrammed into pluripotent stem cells (induced pluripotent stem cells, iPSCs) by overexpression of four Yamanaka factors: Oct4, Sox2, Klf4, and c-Myc (OSKM). Numerous studies have demonstrated that cell fate can be altered by forced expression of these factors, confirming the potential of cell reprogramming as an approach for the treatment of various diseases, and the potential of such approach for anti-aging has also been confirmed.

Partial cell dedifferentiation has been studied as an anti-aging technique using the OSKM. Partial cell dedifferentiation means aged cells and tissues are subjected to partial dedifferentiation reprogramming in vivo without complete dedifferentiation reprogramming, by partially expressing OSKM in vivo for a short period of time, and through this technique, age-related diseases that occur in specific areas can be specifically treated.

However, transient expression of OSKM in vivo has been reported to induce tumorigenesis in various tissues (K. Ohnishi et al., Cell 156, 663-677, 2014), and overexpression of factors such as Klf4 and c-Myc for cell reprogramming has been reported to have a risk of inducing teratoma formation during dedifferentiation (M. Abad et al., Nature 502, 340-345, 2013). Additionally, ectopic Oct4 gene expression in the early stage of dedifferentiation reprogramming induces a promiscuous transcriptome network, leading to off-target reprogramming. Therefore, the cell reprogramming-based anti-aging technique by expression of OSKM in vivo still has significant limitations in terms of stability and efficiency.

Accordingly, there is a need for an efficient and safe anti-aging method using OSKM without directly expressing OSKM in vivo.

### DISCLOSURE

### Technical Problem

Under such circumstances, the present inventors have confirmed that specific proteins derived from plasma directly treated with OSKM in vivo and combinations thereof can prevent, improve or treat an age-related disease by improving age-related phenotypes in vitro and in vivo and improving the symptoms of degenerative brain disease, stroke and liver failure, which are caused by aging, thereby completing the present disclosure.

### Technical Solution

One object of the present disclosure is to provide a pharmaceutical composition for preventing or treating an age-related disease, including a composition, which includes two or more proteins selected from GCLC, MAP1S, ENO1 and KNG1; or a polynucleotide encoding the proteins, as an active ingredient.

Another object of the present disclosure is to provide a pharmaceutical composition for preventing or treating an age-related disease, including a composition, which increases the expression of two or more proteins selected from GCLC, MAP1S, ENO1 and KNG1, as an active ingredient.

Still another object of the present disclosure is to provide an anti-aging method including administering the pharmaceutical composition of the present disclosure to a subject except human.

Still another object of the present disclosure is to provide a composition for inhibiting cell aging, including any one selected from: a composition including two or more proteins selected from GCLC, MAP1S, ENO1, and KNG1 or a polynucleotide encoding the same; or a composition which increases the expression of two or more proteins selected from GCLC, MAP15, ENO1, and KNG1.

### Advantageous Effects

The two or more combinations selected from GCLC, ENO1, Map1s, and KNG1 derived from plasma treated with Oct4, Sox2, Klf4 and c-Myc(OSKM) of the present disclosure improve age-related phenotypes in vitro and in vivo, and improve a disease induced by aging of vascular endothelial cells, a disease induced by aging of organs, a disease induced by aging of nerves, a degenerative brain disease, etc., which are caused by aging, and thus can prevent, improve or treat an age-related disease.

### BRIEF DESCRIPTION OF DRAWINGS

FIG. 1 shows the results of LC/MS analysis for analyzing proteins in plasma (OSKM-treated plasma) derived from 4F2A mice in which OSKM was induced, (a) is a schematic diagram showing the process of screening proteins specifically induced in OSKM-treated plasma. (b-c) are data from LC/MS analysis and protein expression patterns of OSKM-treated plasma. (d) is a list of proteins specifically induced in OSKM-treated plasma.

FIG. 2 shows the results of analyzing age-related markers in cells treated with OSKM-treated plasma or cells in which the expression of GCLC, MAP1S, ENO1, and KNG1 derived from OSKM-treated plasma was induced. (a) is the result of analyzing the expression levels of H3K9me3 and H4k20me3, which are age-related histone markers, after treating cells with OSKM-treated plasma, and (b) is data showing the quantification thereof. (c) is the result of analyzing the expression levels of H3K9me3 and H4k20me3 after inducing the expression of GCLC, MAP1S, ENO1, and KNG1 derived from OSKM-treated plasma in cells, and (d) is data showing the quantification thereof.

FIG. 3 shows the results of quantifying the level of aging cell reduction through aging markers including beta-galactosidase after overexpression of GCLC, MAP1S, ENO1, or KNG1 alone or various combinations thereof in wild-type mouse tail-tip fibroblasts. Additionally, it was confirmed that changes in age-related epigenetic markers as well as a decrease in aging markers were induced when two or more combinations were used among various combinations. For example, as shown in data of FIG. 3c, when GCLC, MAP1S, ENO1, or KNG1 was treated alone, there was no change in the epigenetic marker (H3K9me3), which is known to decrease with aging, but when two or more combinations were treated, a significant increase in the amount of H3K9me3 was observed. In other words, it shows that the level of cell aging can be significantly suppressed and reversed when two or more combinations are used.

FIG. 4 shows the results of analyzing age-related markers in a premature aging mouse model by administration of OSKM-treated plasma or GCLC, MAP1S, ENO1, and KNG1 derived from OSKM-treated plasma. (a-c) are data confirming (a) suppression of weight loss, (b) increase in lifespan, and (c) reduction of spinal curvature after injecting OSKM-treated plasma into a 10-month-old premature aging mouse model. (d-e) are data analyzing stiffness and fibrosis symptoms in the aorta 4 weeks after injection of OSKM-treated plasma in a premature aging mouse model using H&E, VVG, MT, and ORO staining. (f-g) are data analyzing aging symptoms in the liver, spleen, kidney, and skin 4 weeks after injection of OSKM-treated plasma into a premature aging mouse model using H&E staining.

FIG. 5 (a) is data from an open field test performed after injecting OSKM-treated plasma into 6-month-old (young), 18-month-old (mid-aged), and 24-month-old (old, naturally aged) wild-type C57BL′6 mice. (b) is data analyzing the expression levels of age-related genes through qRT-PCR after injecting OSKM-treated plasma into 6-month-old (young) and 24-month-old (old, naturally aged) mice.

FIG. 6 (a) is data analyzing aging symptoms in the liver, spleen, and kidney using H&E staining after injecting a composition containing GCLC, MAP1S, ENO1, and KNG1 into 24-month-old (old, naturally aged) wild-type C57BL/6 mice. (b) is data analyzing stiffness and fibrosis symptoms in the aorta 4 weeks after injection of the composition containing GCLC, MAP1S, ENO1, and KNG1 into 24-month-old wild-type C57BL′6 mice using H&E staining, and (c) shows quantification thereof. (d-e) are data showing the number of cardiac myofibroblasts in the aortic tissue of (d) a 10-month-old premature aging mouse model and (e) 24-month-old wild-type C57BL/6 mice 4 weeks after injection of the composition containing GCLC, MAP1S, ENO1, and KNG1 using α-SMA immunofluorescence staining.

FIG. 7 shows data analyzing the level of (a) Alzheimer's disease-specific amyloid-beta (AP) plaques and (b) Aβ42/40 ratio in brain tissue 2 weeks after injection of the composition containing GCLC, MAP1S, ENO1, and KNG1 derived from OSKM-treated plasma into a 12-month-old Alzheimer's disease mouse model.

FIG. 8 shows data analyzing the mRNA expression levels of (a) Map2, (b) Synapsin, (c) DAT, and (c) TH, which are dopaminergic neuron-specific genes, in midbrain tissue 2 weeks after injection of the composition containing GCLC, MAP1S, ENO1, and KNG1 derived from OSKM-treated plasma into a 12-month-old Parkinson's disease mouse model using qRT-PCR.

FIG. 9 shows data analyzing the (a) infarct volume and (b) neurological deficit score of brain tissue 2 weeks after injection of the composition containing GCLC, MAP1S, ENO1, and KNG1 derived from OSKM-treated plasma into a stroke mouse model.

FIG. 10 shows data analyzing the levels of ATL (alanine transaminase), AST (aspartate transaminase), and ALB (albumin) in plasma 2 weeks after injection of the composition containing GCLC, MAP1S, ENC1, and KNG1 derived from OSKM-treated plasma into a liver failure mouse model.

### DETAILED DESCRIPTION OF PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Additionally, the scope of the present disclosure is not limited by the specific description described below.

One aspect of the present disclosure provides a pharmaceutical composition for preventing or treating an age-related disease, including a composition, which includes two or more proteins selected from GCLC, MAP1S, ENO1 and KNG1; or a polynucleotide encoding the proteins, as an active ingredient

Another aspect of the present disclosure provides a pharmaceutical composition for preventing or treating an age-related disease, including a composition, which increases the expression of two or more proteins selected from GCLC, MAP1S, ENO1 and KNG1, as an active ingredient.

The two or more proteins selected from GCLC, MAP1S, ENO1 and KNG1 of the present disclosure are a combination of 2, 3, or 4 of the above proteins, and specifically, a combination of 2 proteins may include GCLC and ENO1(GE); GCLC and KNG1(GK); GCLC and MAP1S(GM); ENO1 and MAP1S(EM); MAP1S and KNG1(MK); or ENO1 and MAP1S(EK), a combination of 3 proteins may include GCLC, ENO1 and MAP1S(GEM); GCLC, ENO1 and KNG1(GEK); GCLC, MAP1S and KNG1(GMK); or ENO1, MAP15 and KNG1(EMK), and a combination of 4 proteins may include GCLC, MAP1 S, ENO1 and KNG1(GEMK).

Accordingly, one embodiment of the present disclosure provides a pharmaceutical composition for preventing or treating an age-related disease, including a combination of two of the above proteins, that is, a combination selected from GCLC and ENO1(GE); GCLC and KNG1(GK); GCLC and MAP1S(GM); ENO1 and MAP1S(EM); MAP1S and KNG1(MK); and ENO1 and MAP1S(EK), or a polynucleotide encoding the proteins.

Another embodiment of the present disclosure provides a pharmaceutical composition for preventing or treating an age-related disease, including a combination of three of the above proteins, that is, a combination selected from GCLC, ENC1 and MAP1S(GEM); GCLC, ENO1 and KNG1(GEK); GCLC, MAP1S and KNG1(GMK); and ENO1, MAP1S and KNG1(EMK), or a polynucleotide encoding the proteins.

Still another embodiment of the present disclosure provides a pharmaceutical composition for preventing or treating an age-related disease, including a combination of four of the above proteins, that is, a combination of GCLC, MAP1S, ENO1 and KNG1(GEMK), or a polynucleotide encoding the proteins.

As used herein, the term “glutamate-cysteine ligase catalytic subunit (GCLC)” which is a subunit of glutamate-cysteine ligase (GCL), is the rate-limiting enzyme that determines the overall rate of the glutathione (GSH) synthesis pathway.

In the present disclosure, the GCLC protein may have, include, or consist of an amino acid sequence represented by SEQ ID NO: 1, or may consist essentially of the above amino acid sequence. Specifically, the GCLC protein may consist of GenBank Protein ID, 183039. UniProtKB ID, P48506 or a polypeptide represented by the amino acid sequence of SEQ ID NO: 1.

The GCLC protein may be obtained from a known database such as NIH GenBank, etc. For example, in the present disclosure, the amino acid sequence of GCLC protein may be obtained by reference from GenBank Protein ID, 183039. UniProtKB ID, P48506. In another example, the protein may include an amino acid sequence having a homology or identity of at least 70%, 75%, 80%, 85%, 90%, 95%, 96%, 97%, 98%, 99%, 99.5%, 99.7%, or 99.9% to the amino acid sequence represented by SEQ ID NO: 1. Additionally, it is apparent that any protein having an amino acid sequence, in which part of the sequence is deleted, modified, substituted, conservatively substituted or added, may also fall within the scope of the present disclosure as long as the amino acid sequence has such a homology or identity, and exhibits an efficacy corresponding to the protein including GenBank Protein ID, 183039. UniProtKB ID, P48506 or the amino acid sequence of SEQ ID NO: 1.

For example, it may be sequence additions or deletions that do not alter the function of the protein of the present disclosure, naturally occurring mutations, silent mutations therein, or conservative substitutions at the N-terminus, C-terminus, and/or within the amino acid sequences.

As used herein, the term “conservative substitution” refers to substitution of an amino acid with another amino acid having similar structural and/or chemical properties. Such amino acid substitution may generally occur based on similarity of polarity, charge, solubility, hydrophobicity, hydrophilicity, and/or amphipathic nature of a residue. Typically, conservative substitutions may have little or no effect on the activity of a protein or polypeptide.

As used herein, the term ‘homology’ or ‘identity’ refers to the degree of similarity between two given amino acid sequences or nucleotide sequences, and may be expressed as a percentage. The terms homology and identity may often be used interchangeably with each other.

The sequence homology or identity of conserved polynucleotides or polypeptides may be determined by standard alignment algorithms and can be used with a default gap penalty established by the program being used. Substantially, homologous or identical sequences are generally expected to hybridize to all or part of the sequences under moderate or high stringent conditions. It is apparent that hybridization with polynucleotides containing general codon or degenerate codons in hybridizing polynucleotides is also included.

Whether any two polynucleotide or polypeptide sequences have a homology, similarity, or identity may be, for example, determined by a known computer algorithm such as the “FASTA” program (Pearson et al., (1988) [Proc. Natl. Acad. Sci. USA 85]: 2444) using default parameters. Alternatively, it may be determined by the Needleman-Wunsch algorithm (Needleman and Wunsch, 1970, J. Mol. Biol. 48: 443-453), which is performed using the Needleman program of the EMBOSS package (EMBOSS: The European Molecular Biology Open Software Suite, Rice et al., 2000, Trends Genet. 16: 276-277) (preferably, version 5.0.0 or versions thereafter) (GCG program package (Devereux, J., et al., Nucleic Acids Research 12: 387 (1984)), BLASTP, BLASTN, FASTA (Atschul, [S.][F.,][ET AL., J MOLEC BIOL 215]: 403 (1990); Guide to Huge Computers, Martin J. Bishop, [ED.,] Academic Press, San Diego, 1994, and [CARILLO ETA/.](1988) SIAM J Applied Math 48: 1073). For example, the homology, similarity, or identity may be determined using BLAST or ClustalW of the National Center for Biotechnology Information (NCBI).

The homology, similarity, or identity of polynucleotides or polypeptides may be, for example, determined by comparing sequence information using, for example, the GAP computer program, such as Needleman et al. (1970), J Mol Biol. 48: 443 as disclosed in Smith and Waterman, Adv. Appl. Math (1981) 2:482. In summary, the GAP program defines the homology, similarity, or identity as the value obtained by dividing the number of similarly aligned symbols (i.e., nucleotides or amino acids) by the total number of the symbols in the shorter of the two sequences. Default parameters for the GAP program may include (1) a unary comparison matrix (containing a value of 1 for identities and 0 for non-identities) and the weighted comparison matrix of Gribskov et al. (1986), Nucl. Acids Res. 14:6745, as disclosed in Schwartz and Dayhoff, eds., Atlas of Protein Sequence and Structure, National Biomedical Research Foundation, pp. 353-358 (1979) (or EDNAFULL substitution matrix (EMBOSS version of NCBI NUC4.4)); (2) a penalty of 3.0 for each gap and an additional 0.10 penalty for each symbol in each gap (or a gap opening penalty of 10 and a gap extension penalty of 0.5); and (3) no penalty for end gaps.

In the present disclosure, the polynucleotide encoding the GCLC protein may be a Gclc gene.

The polynucleotide encoding the GCLC protein of the present disclosure may include GenBank Protein ID, 183039. UniProtKB ID, P48506 or a nucleotide sequence encoding the amino acid sequence represented by SEQ ID NO: 1. In one example of the present disclosure, the polynucleotide encoding the GCLC protein of the present disclosure may include a sequence identified by Gene ID: 2729 or a nucleotide sequence of SEQ ID NO: 2. Additionally, the polynucleotide encoding the GCLC protein of the present disclosure may consist or consist essentially of the nucleotide sequence of SEQ ID NO: 2. Specifically, the GCLC protein may be encoded by the polynucleotide represented by the nucleotide sequence of SEQ ID NO: 2.

The polynucleotide encoding the Gc/c gene of the present disclosure may undergo various modifications in the coding region within the scope that does not change the amino acid sequence of the GCLC protein, due to codon degeneracy or in consideration of the codons preferred in an organism in which the Gc/c gene of the present disclosure is to be expressed. Specifically, the polynucleotide encoding the GCLC protein of the present disclosure may have or include a nucleotide sequence having a homology or identity of 70% or more, 75% or more, 76% or more, 85% or more, 90% or more, 95% or more, 96% or more, 97% or more, or 98% or more to the nucleotide sequence of SEQ ID NO: 2, or may consist or consist essentially of a nucleotide sequence having a homology or identity of 70% or more, 75% or more, 76% or more, 85% or more, 90% or more, 95% or more, 96% or more, 97% or more, or 98% or more to the nucleotide sequence of SEQ ID NO: 2, but is not limited thereto.

As used herein, the term “microtubule-associated protein S1 (MAP1S)” is a protein that has DNA binding capacity and mediates mitochondrial aggregation that leads to cell death and gene destruction (MAGD) and is known to play a role in anchoring the microtubule organizing center to the centrosome.

In the present disclosure, the MAP1S protein may be obtained from a known database such as NIH GenBank, etc. For example, in the present disclosure, the amino acid sequence of MAP1S protein may be obtained by reference from GenBank: AAH80547.1, UniProt ID Q66K74. In another example, the MAP1S protein may have, include, or consist of an amino acid sequence represented by SEQ ID NO: 3, or may consist essentially of the above amino acid sequence. Specifically, the MAP1S protein may consist of a polypeptide represented by the amino acid sequence of SEQ ID NO: 3.

In the present disclosure, the amino acid sequence of MAP1S protein may include an amino acid sequence having a homology or identity of at least 70%, 75%, 80%, 85%, 90%, 95%, 96%, 97%, 98%, 99%, 99.5%, 99.7%, or 99.9% to the amino acid sequence represented by SEQ ID NO: 3. Additionally, it is apparent that any protein having an amino acid sequence, in which part of the sequence is deleted, modified, substituted, conservatively substituted or added, may also fall within the scope of the present disclosure as long as the amino acid sequence has such a homology or identity, and exhibits an efficacy corresponding to the protein including the amino acid sequence of SEQ ID NO: 3.

In the present disclosure, the polynucleotide encoding the MAP1S protein may be a Map1s gene.

The polynucleotide encoding the MAP1S protein of the present disclosure may include a nucleotide sequence encoding the amino acid sequence represented by GenBank: AAH80547.1, UniProt ID Q66K74 or the amino acid sequence represented by SEQ ID NO: 3. In one example of the present disclosure, the polynucleotide encoding the MAP1S protein of the present disclosure may include a sequence identified by GeneID: 55201 or a nucleotide sequence of SEQ ID NO: 4. Additionally, the polynucleotide encoding the MAP1S protein of the present disclosure may consist or consist essentially of the nucleotide sequence of SEQ ID NO: 4. Specifically, the MAP1S protein may be encoded by the polynucleotide represented by the nucleotide sequence of SEQ ID NO: 4.

The polynucleotide encoding the Map1s gene of the present disclosure may undergo various modifications in the coding region within the scope that does not change the amino acid sequence of the MAP1S protein, due to codon degeneracy or in consideration of the codons preferred in an organism in which the Map1s gene of the present disclosure is to be expressed. Specifically, the polynucleotide encoding the MAP1S protein of the present disclosure may have or include a nucleotide sequence having a homology or identity of 70% or more, 75% or more, 76% or more, 85% or more, 90% or more, 95% or more, 96% or more, 97% or more, or 98% or more to the nucleotide sequence of SEQ ID NO: 4, or may consist or consist essentially of a nucleotide sequence having a homology or identity of 70% or more, 75% or more, 76% or more, 85% or more, 90% or more, 95% or more, 96% or more, 97% or more, or 98% or more to the nucleotide sequence of SEQ ID NO: 4, but is not limited thereto.

As used herein, the term “enolase 1, (ENO1)” refers to an enzyme having activity of converting 2-phosphoglycerate into phosphoenolpyruvate, and is also referred to as alpha-Enolase (a-Enolase).

In the present disclosure, the ENO1 protein may be obtained from a known database such as NIH GenBank, etc. For example, in the present disclosure, the amino acid sequence of ENO1 protein may be obtained by reference from NCBI Accession No: NP_001340275, XP_006710496, and Uniprot ID P06733. In another example, the ENO1 protein may have, include, or consist of an amino acid sequence represented by SEQ ID NO: 5, or may consist essentially of the above amino acid sequence. Specifically, the ENO1 protein may consist of a polypeptide represented by the amino acid sequence of SEQ ID NO: 5.

In the present disclosure, the amino acid sequence of ENO1 protein may include an amino acid sequence having a homology or identity of at least 70%, 75%, 80%, 85%, 90%, 95%, 96%, 97%, 98%, 99%, 99.5%, 99.7%, or 99.9% to NCBI Accession No: NP_001340275, XP_006710496, and Uniprot ID P06733, or the amino acid sequence represented by SEQ ID NO: 5. Additionally, it is apparent that any protein having an amino acid sequence, in which part of the sequence is deleted, modified, substituted, conservatively substituted or added, may also fall within the scope of the present disclosure as long as the amino acid sequence has such a homology or identity, and exhibits an efficacy corresponding to the protein including the amino acid sequence of SEQ ID NO: 5.

In the present disclosure, the polynucleotide encoding the ENO1 protein may be an Eno1 gene.

The polynucleotide encoding the ENO1 protein of the present disclosure may include NCBI Accession No: NP_001340275, XP_006710496, and Uniprot ID P06733 described above, or a nucleotide sequence encoding the amino acid sequence represented by SEQ ID NO: 5. In one example of the present disclosure, the polynucleotide encoding the ENO1 protein of the present disclosure may include a sequence identified by GeneID: 55201 or a nucleotide sequence of SEQ ID NO: 6. Additionally, the polynucleotide encoding the ENO1 protein of the present disclosure may consist or consist essentially of the nucleotide sequence of SEQ ID NO: 6. Specifically, the ENO1 protein may be encoded by the polynucleotide represented by the nucleotide sequence of SEQ ID NO: 6.

The polynucleotide encoding the Eno1 gene of the present disclosure may undergo various modifications in the coding region within the scope that does not change the amino acid sequence of the ENO1 protein, due to codon degeneracy or in consideration of the codons preferred in an organism in which the Eno1 gene of the present disclosure is to be expressed. Specifically, the polynucleotide encoding the ENO1 protein of the present disclosure may have or include a nucleotide sequence having a homology or identity of 70% or more, 75% or more, 76% or more, 85% or more, 90% or more, 95% or more, 96% or more, 97% or more, or 98% or more to the nucleotide sequence of SEQ ID NO: 6, or may consist or consist essentially of a nucleotide sequence having a homology or identity of 70% or more, 75% or more, 76% or more, 85% or more, 90% or more, 95% or more, 96% or more, 97% or more, or 98% or more to the nucleotide sequence of SEQ ID NO: 6, but is not limited thereto.

As used herein, the term “Kininogen 1 (KNG1)” is the precursor protein to low-molecular-weight kininogen, high-molecular-weight kininogen, and bradykinin secreted therefrom. The high-molecular-weight kininogen is known to be an essential element in blood coagulation and in the composition of the kallikrein-kinin system.

In the present disclosure, the KNG1 protein may be obtained from a known database such as NIH GenBank, etc. For example, in the present disclosure, the amino acid sequence of KNG1 protein may be obtained by reference from UniProt ID: P01042. In another example, the KNG1 protein may have, include, or consist of an amino acid sequence represented by SEQ ID NO: 7, or may consist essentially of the above amino acid sequence. Specifically, the ENO1 protein may consist of a polypeptide represented by the amino acid sequence of SEQ ID NO: 7.

In the present disclosure, the amino acid sequence of KNG1 protein may include an amino acid sequence having a homology or identity of at least 70%, 75%, 80%, 85%, 90%, 95%, 96%, 97%, 98%, 99%, 99.5%, 99.7%, or 99.9% to UniProt ID: P01042, or the amino acid sequence represented by SEQ ID NO: 7. Additionally, it is apparent that any protein having an amino acid sequence, in which part of the sequence is deleted, modified, substituted, conservatively substituted or added, may also fall within the scope of the present disclosure as long as the amino acid sequence has such a homology or identity, and exhibits an efficacy corresponding to the protein including the amino acid sequence of SEQ ID NO: 5.

In the present disclosure, the polynucleotide encoding the KNG1 protein may be a Kng1 gene.

The polynucleotide encoding the KNG1 protein of the present disclosure may include UniProt ID: P01042 described above, or a nucleotide sequence encoding the amino acid sequence represented by SEQ ID NO: 7. In one example of the present disclosure, the polynucleotide encoding the KNG1 protein of the present disclosure may include a sequence identified by Gene ID: 3827 or a nucleotide sequence of SEQ ID NO: 8. Additionally, the polynucleotide encoding the KNG1 protein of the present disclosure may consist or consist essentially of the nucleotide sequence of SEQ ID NO: 8. Specifically, the KNG1 protein may be encoded by the polynucleotide represented by the nucleotide sequence of SEQ ID NO: 8.

The polynucleotide encoding the Kng1 gene of the present disclosure may undergo various modifications in the coding region within the scope that does not change the amino acid sequence of the KNG1 protein, due to codon degeneracy or in consideration of the codons preferred in an organism in which the Kng1 gene of the present disclosure is to be expressed. Specifically, the polynucleotide encoding the KNG1 protein of the present disclosure may have or include a nucleotide sequence having a homology or identity of 70% or more, 75% or more, 76% or more, 85% or more, 90% or more, 95% or more, 96% or more, 97% or more, or 98% or more to the nucleotide sequence of SEQ ID NO: 8, or may consist or consist essentially of a nucleotide sequence having a homology or identity of 70% or more, 75% or more, 76% or more, 85% or more, 90% or more, 95% or more, 96% or more, 97% or more, or 98% or more to the nucleotide sequence of SEQ ID NO: 8, but is not limited thereto.

In the present disclosure, the composition which increases the expression of proteins may include a vector containing polynucleotides encoding the proteins or an enhancer which increases the expression of the proteins, etc.

In one example, the composition which increases the expression of two or more proteins selected from GCLC, MAP1S, ENO1 and KNG1 of the present disclosure may be a vector containing two or more polynucleotides selected from the group consisting of a polynucleotide encoding the GCLC protein, a polynucleotide encoding the MAP1S protein, a polynucleotide encoding the ENO1 protein, and a polynucleotide encoding the KNG1 protein. The vector be an individual vector containing each gene, or may contain two or more of the above polynucleotides in a single vector. As used herein, the term “vector” is a DNA capable of introducing a desired DNA fragment into a host cell to be proliferated, and is also referred to as a cloning vehicle. The term “expression vector” means a recombinant DNA molecule including a desired coding sequence and other appropriate nucleotide sequences that are essential for the expression of the operably-linked coding sequence in a specific host organism. In the present disclosure, the vector and the expression vector may be used as the same meaning.

The vector used in the present disclosure is not particularly limited as long as it can be replicated in a host cell, and any vector known in the art may be used. Examples of the vector typically used may include natural or recombinant plasmids, cosmids, viruses, and bacteriophages. For example, as a phage vector or cosmid vector, pWE15, M13, MBL3, MBL4, IXII, ASHII, APII, t10, t11, Charon4A, and Charon21A, etc. may be used; as a plasmid vector, those based on pDZ, pBR, pUC, pBluescriptII, pGEM, pTZ, pCL and pET, etc. may be used; and as a viral vector, adeno-associated virus (AAV) vector, adenovirus vector, herpes virus vector, retrovirus vector, lentivirus vector, vaccinia virus vector, poxvirus vector, herpes simplex virus vector, etc. may be used.

The insertion of the polynucleotide into the chromosome may be performed by any method known in the art, for example, by homologous recombination, but is not limited thereto.

As used herein, the term “transformation” refers to the introduction of a recombinant vector containing a polynucleotide encoding a target protein into a host cell so that the protein encoded by the polynucleotide can be expressed in the host cell. As long as the transformed polynucleotide can be expressed in the host cell, it does not matter whether the transformed polynucleotide is integrated into the chromosome and located therein, or located extrachromosomally, and both cases can be included.

The transformation method may include any method for introducing a nucleic acid into a cell, and suitable standard techniques known in the art may be selected and used depending on the host cell. For example, the method may include electroporation, CaPO₄ precipitation, CaCl₂ precipitation, microinjection, a polyethylene glycol (PEG) method, a DEAE-dextran method, a cationic liposome method, and a lithium acetate-DMSO method, etc., but is not limited thereto.

As used herein, the term “operably linked” means that the polynucleotide sequence is functionally linked to a promoter sequence or an expression regulatory region that initiates and mediates transcription of the polynucleotide encoding the target protein of the present disclosure. The operable linkage may be prepared using a genetic recombinant technology known in the art, and site-specific DNA cleavage and ligation may be prepared using cleavage and ligating enzymes, etc., known in the art, but is not limited thereto.

In the present disclosure, the composition including two or more proteins selected from GCLC, MAP1 S, ENO1, and KNG1 or a polynucleotide encoding the proteins; or the composition which increases the expression of two or more proteins selected from GCLC, MAP1S, ENO1, and KNG1 may increase the endogenous expression and/or activity of two or more proteins selected from GCLC, MAP1S, ENO1, and KNG1 as compared to the endogenous expression and/or activity of two or more proteins selected from wild-type GCLC, MAP1S, ENO1, and KNG1.

As used herein, the term “aging” includes a concept encompassing degenerative changes that occur due to the deterioration of the body structures and functions with age, and changes due to aging are very diverse, such as reductions in each tissue weight and body weight resulting from a decrease in the number of parenchymal cells, a change in connective tissues, a change in body composition, reduction in elasticity of blood vessels or skin, etc., decline in the function of each organ, reduction in disease recovery ability including immunity, decline in the function of sensory organ, and deterioration in memory, learning ability, and comparison ability, etc.

As used herein, the term “inhibition of aging” refers to any action that inhibits, suppresses, improves, or delays the above-described aging symptoms by administration of the composition of the present disclosure, and specifically means any action that at least reduces parameters associated with the above-described aging, for example, severity of a symptom, and encompasses any action that alleviates, relieves, or favorably changes aging symptoms by administration of the composition of the present disclosure. The term “inhibition of aging” may be used interchangeably with “anti-aging”. The “inhibition of aging” may also be expressed as “treatment of aging”, and may also include the treatment and/or improvement of an age-related disease.

As used herein, the term “age-related disease” may include a disease inducing premature aging or a disease induced by aging

In the present disclosure, the disease inducing premature aging may include, for example, Hutchinson Guilford progeria syndrome (HGPS), premature aging associated with HIV infection, muscular dystrophy, Charcot-Marie-Tooth disease, Werner syndrome, insulin-resistant type II diabetes, osteoporosis, skin aging, and restrictive dermopathy, etc, but is not limited thereto, and may include any disease including pathological features of premature aging. Specifically, the disease inducing premature aging may be Hutchinson Guilford progeria syndrome, but is not limited thereto.

In the present disclosure, the disease induced by aging may include, for example, arteriosclerosis, liver failure, stroke, degenerative brain disease, Alzheimer's disease, Parkinson's disease, hypertension, cognitive dysfunction, sarcopenia, dry eye, macular degeneration, hyperopia, myopia, cataracts, tinnitus, hearing loss, indigestion, diarrhea, autoimmune disease, pneumonia, influenza, tetanus, infective endocarditis, cancer, overactive bladder, urinary incontinence, benign prostatic hyperplasia, lower urinary tract symptoms, glomerulonephritis, chronic renal failure, osteoporosis, arthritis, diabetes, renal failure, chronic obstructive pulmonary disease, and pulmonary fibrosis, but is not limited thereto, and may include any disease including pathological features caused by aging. Specifically, the disease induced by aging may be a disease induced by aging of vascular endothelial cells, a disease induced by aging of organs, a disease induced by aging of nerves, a degenerative brain disease, etc., and more specifically, it may be one or more selected from arteriosclerosis, liver failure, stroke, Alzheimer's disease, and Parkinson's disease, but is not limited thereto.

As used herein, the term “prevention” means all of the actions to inhibit or delay the onset of age-related disease by administration of the composition of the present disclosure, and the term “treatment” means all of the actions to improve or beneficially change the symptoms of age-related disease by administration of the composition of the present disclosure

As used herein, the term “improvement” means all of the actions to improve or beneficially change the symptoms in an individual who is suspected of having age-related disease or who has developed the same.

The pharmaceutical composition of the present disclosure may further include an appropriate carrier, excipient, or diluent commonly used in the preparation of the pharmaceutical composition. In particular, the amount of the active ingredient included in the pharmaceutical composition may be, but is not particularly limited to, 0.0001% by weight to 10% by weight, preferably 0.001% by weight to 1% by weight, based on the total weight of the composition.

The pharmaceutical composition may have any one formulation selected from the group consisting of tablets, pills, powders, granules, capsules, suspensions, solutions for internal use, emulsions, syrups, sterilized aqueous solutions, non-aqueous solvents, suspensions, emulsions, lyophilized preparations, and suppositories, and may have various formulations for oral or parenteral administration. When formulated, the composition of the present disclosure may be prepared using commonly used diluents or excipients, such as fillers, extenders, binders, wetting agents, disintegrants, surfactants, etc. Solid formulations for oral administration include tablets, pills, powders, granules, capsules, etc., and such solid formulations are prepared by mixing one or more compounds with one or more excipients, for example, starch, calcium carbonate, sucrose, lactose, gelatin, etc. In addition to simple excipients, lubricants such as magnesium stearate, talc, etc. may also be used. Liquid formulations for oral administration include suspensions, solutions for internal use, emulsions, syrup, etc., and may include various excipients, for example, wetting agents, flavoring agents, aromatics, preservatives, etc., in addition to water and liquid paraffin, which are simple diluents frequently used. Formulations for parenteral administration include sterilized aqueous solutions, non-aqueous solutions, suspensions, emulsions, lyophilized preparations, and suppositories. As non-aqueous solvents or suspending agents, propylene glycol, polyethylene glycol, plant oils such as olive oil, injectable esters such as ethyl oleate, etc. may be used. As a base of suppositories, witepsol, Macrogol. Tween 61, cacao butter, laurin fat, glycerogelatin, etc. may be used.

The pharmaceutical composition of the present disclosure may further include other substances that prevent or treat an age-related disease. For example, the pharmaceutical composition of the present disclosure may further include OSKM, a vector expressing OSKM, or plasma treated with OSKM.

The pharmaceutical composition of the present disclosure may be administered in a pharmaceutically effective amount.

As used herein, the term “pharmaceutically effective amount” refers to an amount sufficient to treat diseases at a reasonable benefit/risk ratio applicable to any medical treatment. The effective dosage level may be determined depending on factors including a subject's type and severity, age and sex, the type of disease, the activity of a drug, sensitivity to the drug, the administration time and route, excretion rate, the duration of treatment, drugs used concurrently, and other factors known in the medical field. The pharmaceutical composition of the present disclosure may be administered individually or in combination with other therapeutic agents, and may be administered sequentially or simultaneously with traditional therapeutic agents. The composition may be administered in a single- or multiple-dosage form. It is important to administer the pharmaceutical composition in the minimum amount that exhibits the maximum effect without causing side effects, in view of all the above-described factors, and this amount may be easily determined by those skilled in the art. A preferred administration dosage of the pharmaceutical composition of the present disclosure may vary depending on a patient's condition, body weight, severity of disease, the type of a drug, administration route, and period. However, for preferred effects, the pharmaceutical composition of the present disclosure may be administered at a daily dose of 0.0001 mg/kg to 500 mg/kg, preferably 0.001 mg/kg to 100 mg/kg. The pharmaceutical composition may be administered once or several times a day. The pharmaceutical composition may be administered to various mammals such as rats, livestock, humans, etc. via various routes. All modes of administration common in the art are included without limitation, and may be administered, for example, by orally, rectally, or by intravenous, intramuscular, subcutaneous, intrauterine, or intracerebroventricular injection.

Additionally, the pharmaceutical composition of the present disclosure may be used in the form of veterinary medicines as well as medicines applied to humans. In particular, the animal is a concept including livestock and pets.

In the present disclosure, the composition including two or more proteins selected from GCLC, MAP1 S, ENO1, and KNG1 or a polynucleotide encoding the proteins; or the composition which increases the expression of two or more proteins selected from GCLC, MAP1S, ENO1, and KNG1 may have a cell aging inhibitory effect.

The cell aging inhibitory effect of the present disclosure may be achieved by the endogenous expression and/or activity of two or more proteins selected from GCLC, MAP1S, ENO1, and KNG1 according the treatment of the composition provided in the present disclosure.

The cell aging inhibitory effect may be any one or more effects selected from the following:
i) reduction in the expression level of age-related genes;
ii) reduction in the level of DNA double-strand breaks; and
iii) reduction in the number of beta-galactosidase positive cells.

The i) age-related gene may be any one or more selected from the group consisting of a stress response gene, an extracellular matrix (ECM)-remodeling gene, a senescence-associated secretory phenotype (SASP) gene, and an age-related gene.

The stress response gene may be, for example, Ccl8, p16, p21, Btg2, and Atf, etc., but is not limited thereto.

The ECM-remodeling gene may be, for example, MMP12, MMP13, and IL-6, etc., but is not limited thereto.

The SASP genes may be, for example, Cdkn2a, Cdkn1a, and IL-a, etc., but is not limited thereto.

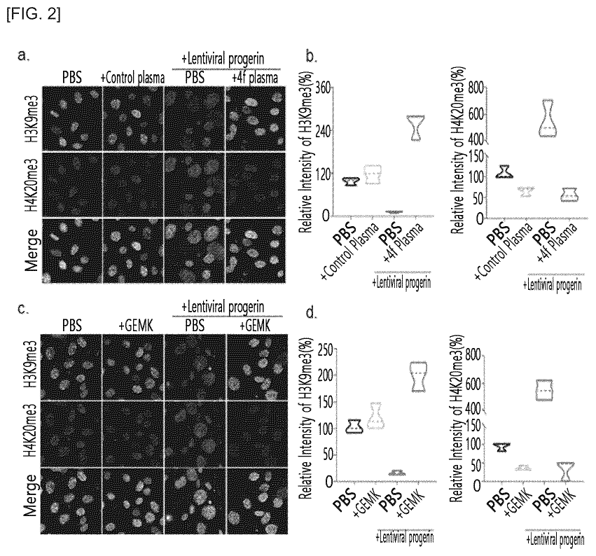

With respect to i) reduction in the expression level of age-related genes, in one embodiment of the present disclosure, the expression levels of age-related genes, Cc18, IL-6, Cdkn2a, Btg2, Atf3, MMP12, MMP13, p16, and p21, were significantly increased in aged fibroblasts, but were restored by the introduction of the dCas9-activator vector (FIG. 2e).

From the above results, it can be seen that the dCas9-activator vector can effectively improve senescence-associated molecular phenotypes by activating endogenous Oct4 activation in aged fibroblasts.

With respect to ii) reduction in the level of DNA double-strand breaks, in one embodiment of the present disclosure, to evaluate the level of DNA double-strand breaks associated with cell aging in aged mouse fibroblasts transduced with mutant laminA/C (LMNA), which induced the expression of GCLC, MAP1S, ENO1, and KNG1, the expression levels of histone markers, H3K9me3 and H4K20me3, were analyzed. When the expression of GCLC, MAP1S, ENO1, and KNG1 derived from OSKM-treated plasma was induced (FIGS. 2c-d), it was confirmed that the expression level of H3K9me3 was increased and the expression level of H4K20me3 was decreased, thereby improving the age-related markers.

In particular, when GCLC, MAP1S, ENO1, or KNG1 was treated alone, there was no change in the epigenetic marker (H3K9me3), which is known to decrease with aging, but when a combination of two or more proteins was treated, a significant increase in the amount of H3K9me3 was observed. This result indicates that when a combination of two or more of the four proteins is used, the level of cell aging can be significantly suppressed.

With respect to iii) reduction in the number of beta-galactosidase positive cells, in one embodiment of the present disclosure, vectors expressing GCLC, MAP1S, ENO1 or KNG1 alone, or various combinations thereof [GCLC+ENO1(GE), GCLC+KNG1(GK), GCLC+MAP1S(GM), ENO1+MAP1S(EM), MAP1S+KNG1(MK), ENO1+MAP1S(EK), GCLC+ENO1+MAP1S(GEM), GCLC+ENO1+KNG1(GEK), GCLC+MAP1 S+KNG1(GMK), ENO1+MAP1 S+KNG1 (EMK), GCLC+MAP1S+ENO1+KNG1(GEMK)] were transformed into wild-type mouse tail-tip fibroblasts, and the number of age-related beta-galactosidase-positive cells was measured after 3 days.

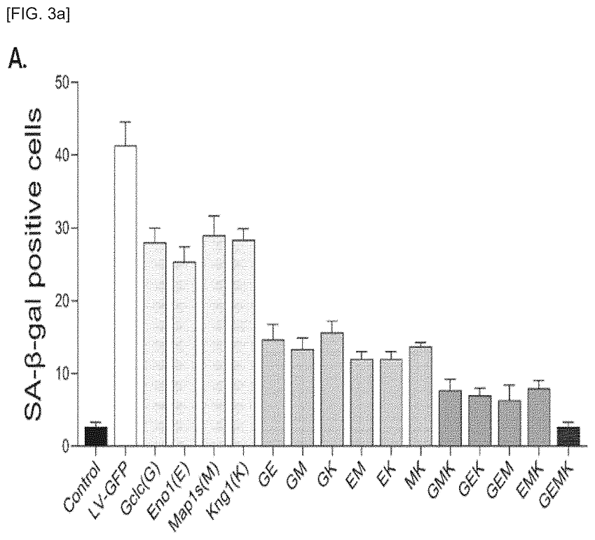

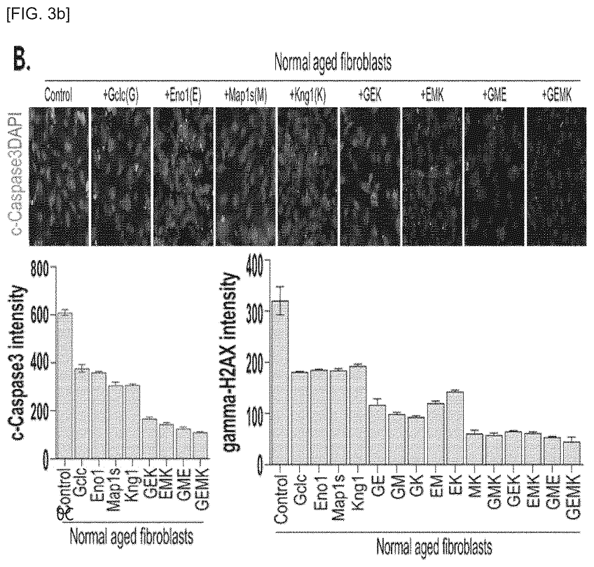

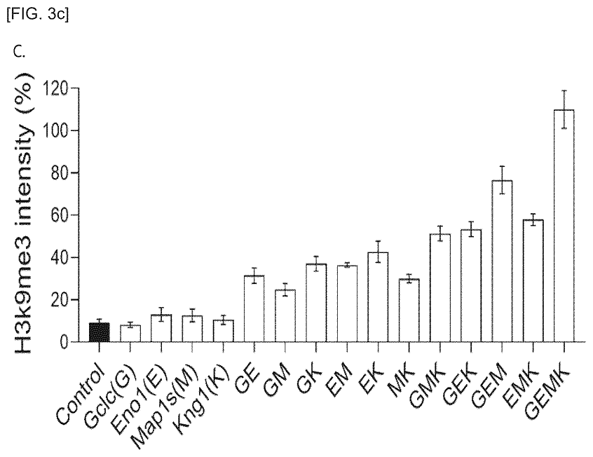

As a result, when GCLC, MAP1S, ENO1, or KNG1 was treated alone, the number of beta-galactosidase-positive cells was reduced, whereas when two or more thereof were treated in combination, the effect of reducing beta-galactosidase-positive cells was more superior, and when GCLC+MAP1S+ENO1+KNG1 (GEMK) was treated, the effect of reducing beta-galactosidase-positive cells was the most superior, confirming that the level of reduction was similar to that of the control wild-type mouse fibroblasts (FIG. 3).

From the above results, it was confirmed that when the expression of GCLC, MAP1 S, ENO1, or KNG1 alone or in combination was induced, age-related markers were improved in cells, confirming the aging inhibitory effect.

In particular, when two or more of GCLC, MAP1S, ENO1, and KNG1 proteins were expressed in combination, age-related markers were improved in cells compared to when GCLC, MAP1S, ENO1, or KNG1 was expressed alone, and when three or four proteins were expressed in combination, age-related markers were significantly improved, confirming that the aging inhibitory effect was significant in combinations of proteins including two or more of GCLC, MAP1, ENO1, and KNG1.

In the present disclosure, the composition including GCLC, MAP1S, ENO1, and KNG1 proteins of the present disclosure; or the composition which increases the expression of GCLC, MAP1S, ENO1, and KNG1 proteins may have an effect of improving an age-related disease.

The effect of improving an age-related disease may be any one or more effects selected from the following:
i) reduction of spinal curvature;
ii) suppression of weight loss;
iii) increase in life span;
iv) increase in motor activity; and
v) improvement of aging of organs and skin.

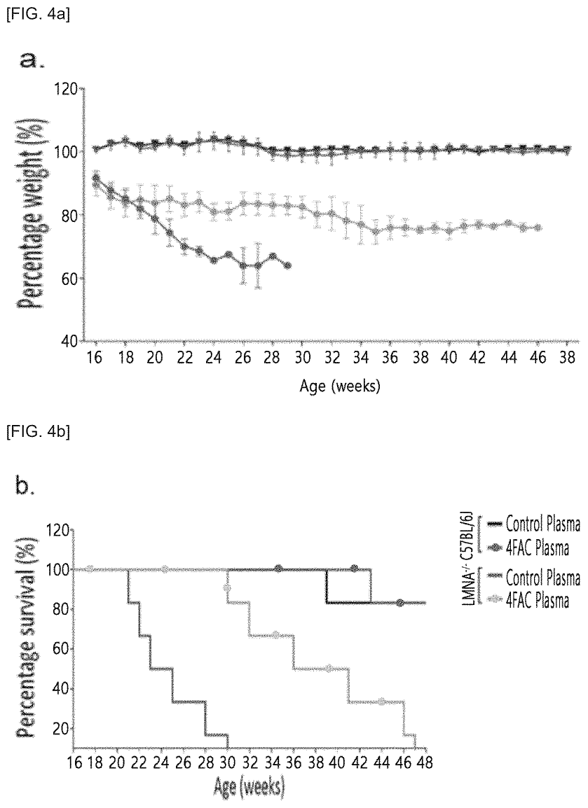

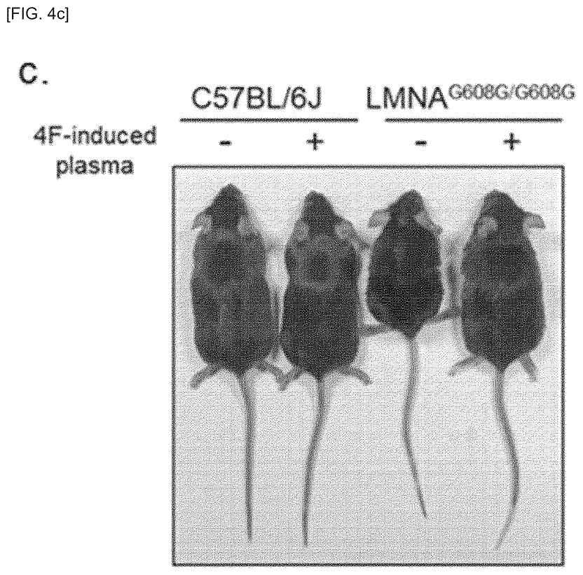

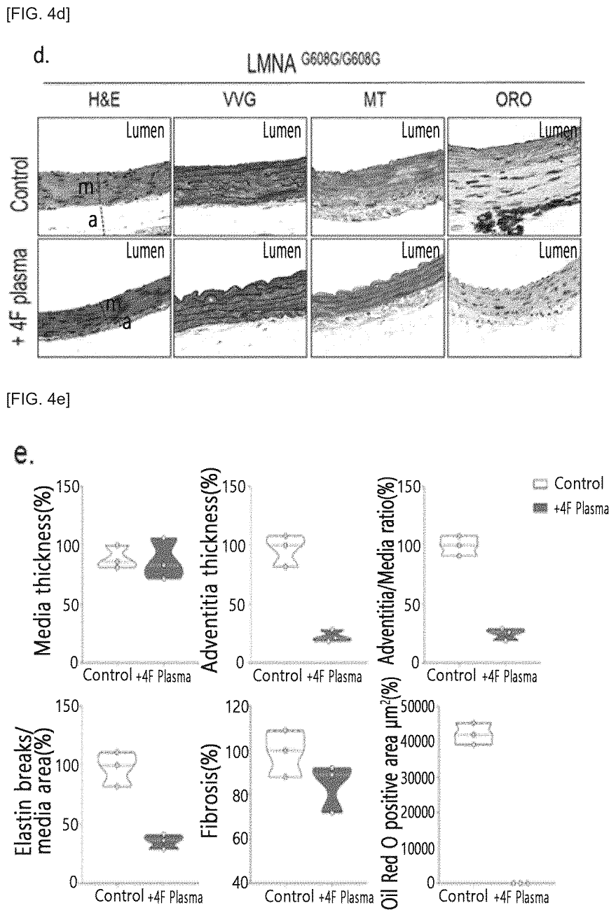

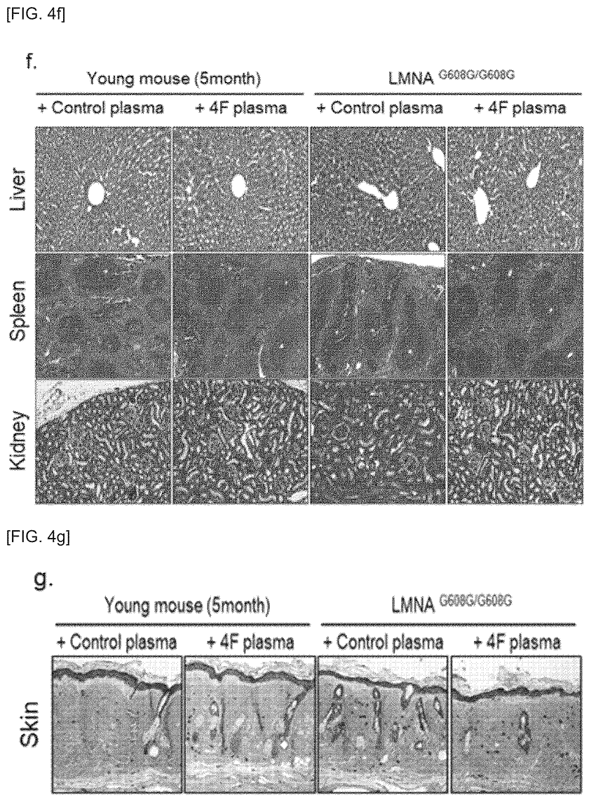

In one embodiment of the present disclosure, when OSKM-treated plasma containing GCLC, MAP1 S, ENO1, and KNG1 was injected into a premature aging mouse model, the appearance was significantly improved, including a reduction in spinal curvature compared to the control group (FIG. 4c), weight loss was suppressed (FIG. 4a), and lifespan was increased (FIG. 4b).

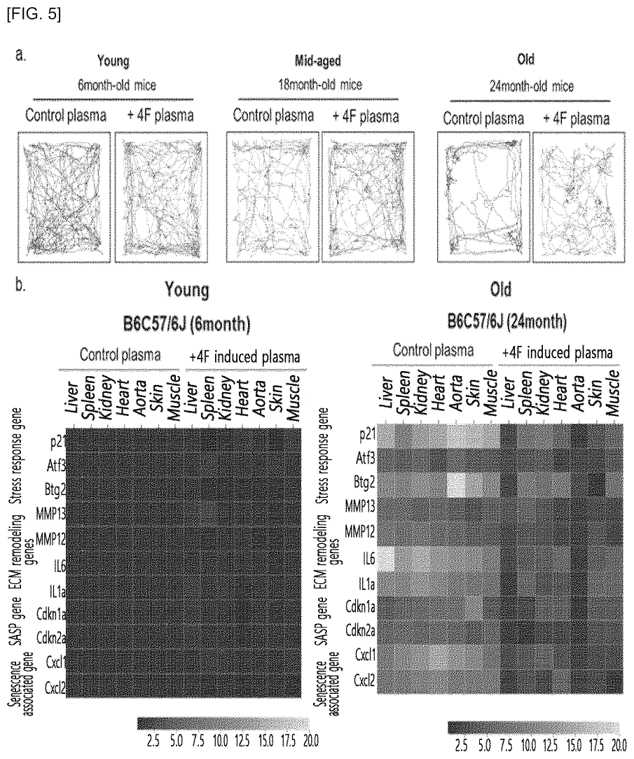

In another embodiment of the present disclosure, the OSKM-treated plasma containing GCLC, MAP1S, ENO1, and KNG1 was injected into 6-month-old (young), 18-month-old (mid-aged), and 24-month-old (old, naturally aged) mice, and then an open field test was performed. As a result, the mice showed increased motor activity compared to the control group after the injection (FIG. 5a).

In still another embodiment of the present disclosure, the OSKM-treated plasma containing GCLC, MAP1, ENO1, and KNG1 was injected into a premature aging mouse model, and after 4 weeks, the aging symptoms in the liver, spleen, kidney, and skin were analyzed H&E staining. As a result, the aging symptoms in the organ and skin were improved in the mice after the injection, compared to the control group (5-month-old mice) (FIG. 4f-g).

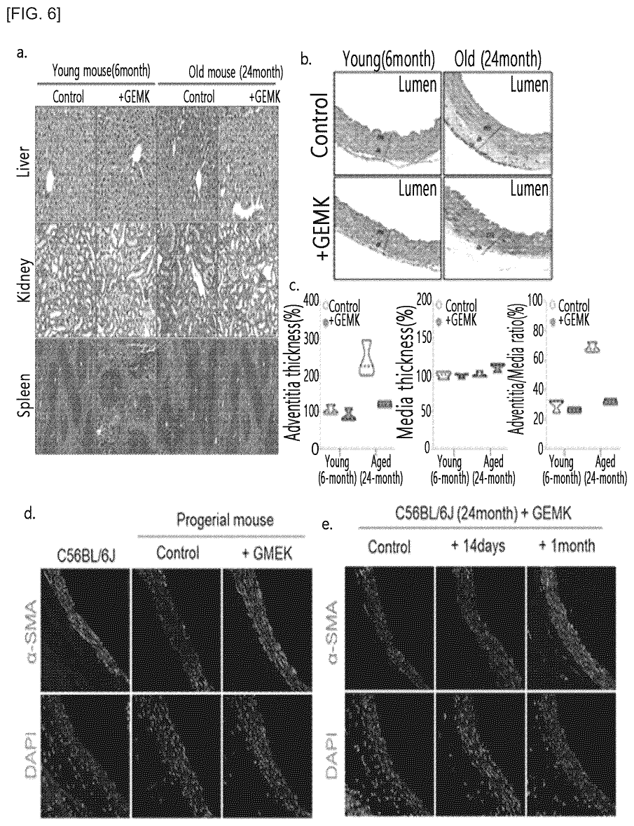

Additionally, the composition including GCLC, MAP1S, ENO1, and KNG1 was injected into naturally aged 24-month-old wild-type C57BL/6 mice, and then the aging symptoms in the liver, spleen, and kidney were analyzed in the same manner. As a result, the aging symptoms in the organ were improved in the mice after the injection, compared to the control group (6-month-old mice) (FIG. 6a).

In still another embodiment of the present disclosure, the composition including GCLC, MAP15, ENO1, and KNG1 was injected into a premature aging mouse model and 24-month-old wild-type C57BL/6 mice, and after 4 weeks, the number of cardiac myofibroblasts in the heart aortic tissue was analyzed through α-SMA immunofluorescence staining. As a result, it was confirmed that the number of α-SMA-positive cells was increased in the mice after the injection, compared to the control group, indicating an increase in the number of cardiac myofibroblasts (FIGS. 6d-e).

Additionally, the composition including GCLC, MAP1S, ENO1, and KNG1 was injected into a premature aging mouse model and 24-month-old wild-type C57BL/6 mice, and after 4 weeks, the number of cardiac myofibroblasts in the aortic tissue was analyzed using α-SMA immunofluorescence staining. As a result, it was confirmed that the number of α-SMA-positive cells was increased in the mice after the injection, compared to the control group, indicating an increase in the number of cardiac myofibroblasts (FIGS. 6d-e).

In the present disclosure, the age-related disease may include a disease inducing premature aging or a disease induced by aging.

The disease inducing premature aging is as described above, and specifically, it may be Hutchinson-Gilford progeria syndrome, but is not limited thereto.

The disease induced by aging is as described above, and specifically, the disease induced by aging may be a disease induced by aging of vascular endothelial cells, a disease induced by aging of organs, a disease induced by aging of nerves, a degenerative brain disease, etc., and more specifically, it may be arteriosclerosis, liver failure, stroke, Alzheimer's disease, and Parkinson's disease, but is not limited thereto.

In any one of the above-described embodiments, the composition including two or more proteins selected from GCLC, MAP1S, ENO1, and KNG1 of the present disclosure or a polynucleotide encoding the same: or the composition which increases the expression of two or more proteins selected from GCLC, MAP1S, ENO1, and KNG1 may have an effect of improving a disease induced by aging of vascular endothelial cells.

The disease induced by aging of vascular endothelial cells may specifically be arteriosclerosis, but is not limited thereto.

The improvement of arteriosclerosis may be achieved by the effect of reducing stiffness of the aorta and/or reducing fibrosis of the aorta.

In one embodiment of the present disclosure, the OSKM-treated plasma containing GCLC, MAP1S, ENO1, and KNG1 was injected into a premature aging mouse model, and after 4 weeks, stiffness and fibrosis symptoms in the aorta were analyzed using H&E, VVG, MT, and ORO staining. As a result, it showed that the stiffness and fibrosis symptoms in the aorta were improved in the mice after the injection, compared to the control group (FIGS. 4d-e).

Additionally, the composition including GCLC, MAP1S, ENO1, and KNG1 was injected into naturally aged 24-month-old wild-type C57BL/6 mice, and after 4 weeks, stiffness and fibrosis symptoms in the aorta were analyzed using H&E staining. As a result, it showed that the stiffness and fibrosis symptoms in the mice after injection were improved as compared to the control group (6-month-old mice) (FIGS. 6b-c).

In any one of the above-described embodiments, the composition including GCLC, MAP1S, ENO1, and KNG1 proteins of the present disclosure; or the composition which increases the expression of GCLC, MAP1S, ENO1, and KNG1 proteins may have an effect of improving a disease induced by aging of organs.

The disease induced by aging of organs may specifically be liver failure, but is not limited thereto.

The improvement of liver failure may be achieved by any one or more effects selected from the following:
i) reduction in the level of alanine transaminase (ATL);
ii) reduction in the level of aspartate transaminase (AST); and
iii) reduction in the level of plasma albumin (ALB).

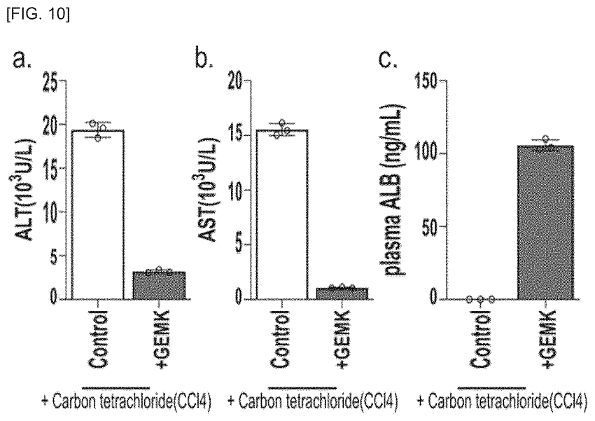

In one embodiment of the present disclosure, the composition including GCLC, MAP1S, ENO1, and KNG1 derived from OSKM-treated plasma was injected into a liver failure mouse model, and after 2 weeks, the plasma proteins were analyzed. As a result, the levels of alanine transaminase (ATL) and aspartate transaminase (AST) were decreased and the level of plasma ALB (albumin) was increased in the mice after the injection, compared to the control group, confirming that the administration of the composition including GCLC, MAP1S, ENO1, and KNG1 improved the symptoms of liver failure (FIG. 10).

In any one of the above-described embodiments, the composition including GCLC, MAP1 S, ENO1, and KNG1 proteins of the present disclosure; or the composition which increases the expression of GCLC, MAP1S, ENO1, and KNG1 proteins may have an effect of improving a disease induced by aging of nerves.

The disease induced by aging of organs may specifically be stroke, but is not limited thereto.

The improvement of stroke may be achieved by the effect of reducing infarct volume and/or reducing neurological deficit score.

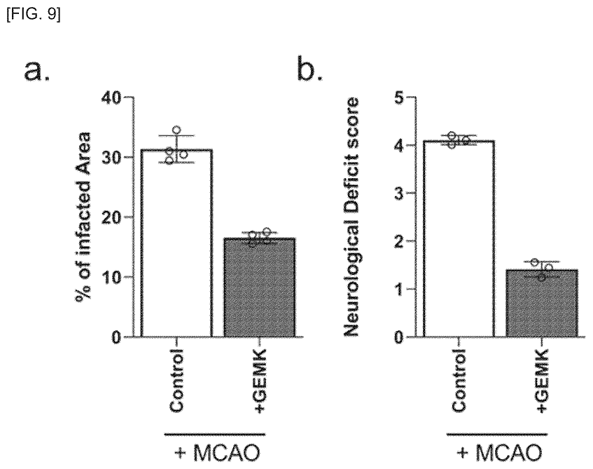

In one embodiment of the present disclosure, the composition including GCLC, MAP1S, ENO1 and KNG1 derived from OSKM-treated plasma was injected into a stroke mouse model, and after 2 weeks, the brain tissue was analyzed for neurology. As a result, it was confirmed that the infarct volume and neurological deficit score were reduced in the mouse after the injection, compared to the control group, thereby confirming that the administration of the composition including GCLC, MAP1S, ENO1 and KNG1 improved the symptoms of stroke (FIG. 9).

In any one of the above-described embodiments, the composition including GCLC, MAP1S, ENO1, and KNG1 proteins of the present disclosure; or the composition which increases the expression of GCLC, MAP1S, ENO1, and KNG1 proteins may have an effect of improving a degenerative brain disease.

The degenerative brain disease may specifically be Alzheimer's disease or Parkinson's disease, but is not limited thereto.

The improvement of Alzheimer's disease or Parkinson's disease may be achieved by reduction in the level of amyloid-beta (Aβ) plaques and Aβ42/40 ratio, or reduction in the expression levels of Map2, Synapsin, DAT, and TH, which are dopaminergic neuron-specific genes.

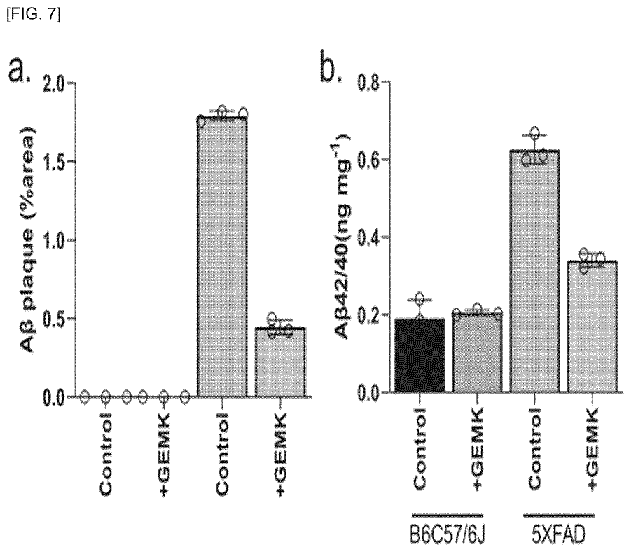

In one embodiment of the present disclosure, the composition including GCLC, MAP1S, ENO1, and KNG1 derived from OSKM-treated plasma was injected into a 12-month-old Alzheimer's disease mouse model, and after 2 weeks, the levels of Alzheimer's disease-specific amyloid-beta (Aβ) plaques and Aβ42/40 ratios in brain tissues were analyzed. As a result, the levels of Aβ plaques and Aβ42/40 ratio were reduced in the mice after the injection compared to the control group, confirming that administration of the composition including GCLC, MAP1S, ENO1, and KNG1 improved the symptoms of Alzheimer's disease (FIG. 7).

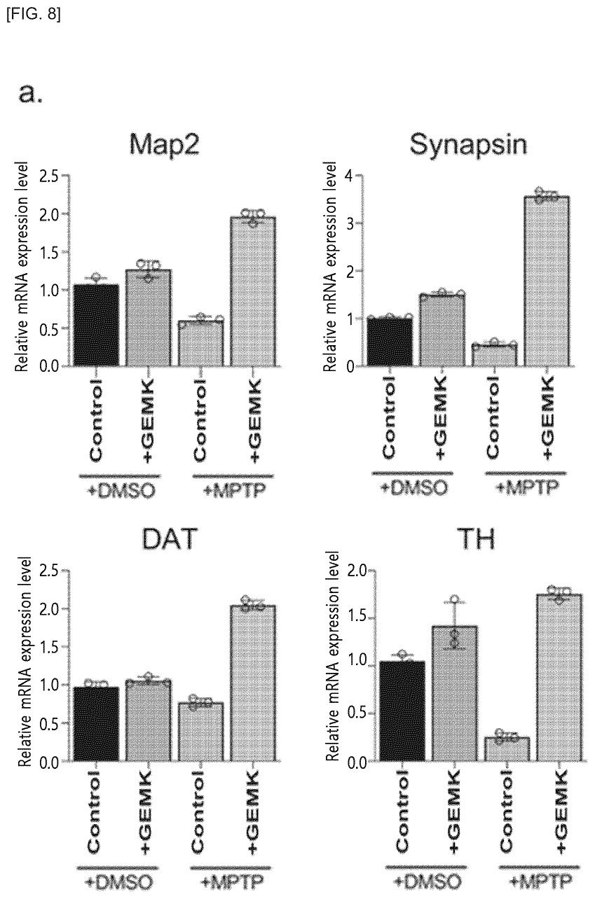

In another embodiment of the present disclosure, the composition including GCLC, MAP1S, ENO1 and KNG1 derived from OSKM-treated plasma was injected into a 12-month-old Parkinson's disease mouse model, and after 2 weeks, the mRNA expression levels of Map2, Synapsin, DAT and TH, which are dopaminergic neuron-specific genes, were analyzed in the midbrain tissues. As a result, the mRNA expression levels of Map2, Synapsin, DAT and TH, which are dopaminergic neuron-specific genes, were increased in the mice after the injection compared to the control group, confirming that the administration of the composition including GCLC, MAP1S, ENO1 and KNG1 improved the symptoms of Parkinson's disease (FIG. 8).

Still another aspect of the present disclosure provides an anti-aging method, including administering the pharmaceutical composition of the present disclosure to a subject.

The terms used in this aspect are as described above.

The method of the present disclosure may be a method for preventing or treating a disease inducing premature aging or a disease induced by aging.

As described above, since the composition including GCLC, MAP1S, ENO1, and KNG1 proteins of the present disclosure; or the composition which increases the expression of GCLC, MAP1S, ENO1, and KNG1 proteins may have an aging inhibitory effect and/or an effect of improving a disease inducing premature aging or a disease induced by aging, the pharmaceutical composition including the composition may be used for the purpose of preventing or improving aging, or preventing, improving or treating a disease inducing premature aging or a disease induced by aging.

The disease inducing premature aging may be, but is not limited to, Hutchinson-Gilford progeria syndrome. This is as described above.

The disease induced by aging may be a disease induced by aging of vascular endothelial cells, a disease induced by aging of organs, a disease induced by aging of nerves, a degenerative brain disease, etc., and more specifically, it may be arteriosclerosis, liver failure, stroke, Alzheimer's disease, and Parkinson's disease, but is not limited thereto. These are as described above.

As used herein, the term “subject” may refer to any animal including humans. The animal may be not only humans but also mammals, such as cows, horses, sheep, pigs, goats, camels, antelopes, dogs, cats, etc. in need of treatment for a similar symptom to the human. Additionally, the subject may refer to animals excluding humans, but is not limited thereto.

As used herein, the term “administration” means introducing the pharmaceutical composition of the present disclosure to a subject suspected of having an age-related disease by any suitable method. The pharmaceutical composition of the present disclosure may be administered through various routes of oral or parenteral administration as long as the pharmaceutical composition can reach a target tissue.

The pharmaceutical composition of the present disclosure may be administered in a pharmaceutically effective amount, as described above.

The pharmaceutical composition of the present disclosure may be applied to any subject without particular limitation as long as the pharmaceutical composition has a purpose of preventing or treating an age-related disease. For example, the pharmaceutical composition may be applied to non-human animals, such as monkeys, dogs, cats, rabbits, marmots, rats, mice, cows, sheep, pigs, and goats, birds, fish, etc., and the pharmaceutical composition may be administered through parenteral, subcutaneous, intraperitoneal, intrapulmonary, and intranasal routes, and for topical treatment, if necessary, the pharmaceutical composition may be administered by any suitable method including intralesional administration. A preferable dose of the pharmaceutical composition of the present disclosure may vary depending on condition and body weight of a subject, severity of the disease, the dosage form of the drug, and the mode and duration of administration, but may be appropriately selected by those skilled in the art. For example, the pharmaceutical composition may be administered by oral, rectal, intravenous, intramuscular, subcutaneous, intrauterine, or intracerebroventricular injection, but is not limited thereto.

The pharmaceutical composition may further include other substances that prevent or treat an age-related disease. For example, the pharmaceutical composition of the present disclosure may further include OSKM, a vector expressing OSKM, or a plasma treated with OSKM.

In another example, the composition provided in the present disclosure may further include one or more ingredients selected from Fgf17, Gpld1, GDF11, superoxide dismutase (SOD), Catalase (CAT), Glutathione(GSH), Glutathione peroxidase(GPx), oxidized glutathione (GSSG). Glutathione S-transferase (GST), Ascorbic acid (vitamin C), α-tocopherol (vitamin E), Folic acid (vitamin B), Polyphenols, carotenoids, Flavonoids, Phenolic Acids, Ferulic acid, Tannin, Catechinic acid, Rosmarinic acid, Apiolin, Hesperetin, Agathisflavone, Monodehydroascorbate reductase (MDHAR), Dehydroscorbate reductase (DHAR), Ascorbate peroxidase (APx), Glutathione reductase (GR), Albumins, Lactoferrin, Metallothioneins, Polyamines, Vitamin K, Ubiquinone, Zinc, Selenium, copper (Cu), iron (Fe) and manganese (Mn), selenium (Se)Allium, Allyl sulfides, Coenzyme Q10, Uric acid, Hydroxycinnamic acid, Hydroxybenzoic acid, lycopene, zeaxanthine, proanthocyanidins, Albumin, Ceruloplasmin, Ferritin, Myoglobin, Transferrin, NADPH, NADH, Lipoic acid, FGF21, insulin-like growth factor-1 (IGF-1), Nicotinamide mononucleotide (NMN), Resveratrol, Metformin, Vitamin D3, Statin, Aspirin, Alpha lipoic acid (ALA), Spermidine, Quercetin, Fisetin, and TMG (trimethylglycine or betaine) Still another aspect of the present disclosure provides a health functional food composition for preventing or treating an age-related disease, including any one selected from a composition including two or more proteins selected from GCLC, MAP1S, ENO1 and KNG1, or a polynucleotide encoding the proteins; or a composition which increases the expression of two or more proteins selected from GCLC, MAP1S, ENO1 and KNG1.

The terms used in this aspect are as described above.

The composition of the present disclosure may have a purpose of preventing or improving a disease inducing premature aging or a disease induced by aging.

As described above, since the composition provided in the present disclosure may have an aging inhibitory effect and/or an effect of improving a disease inducing premature aging or a disease induced by aging, the pharmaceutical composition including the composition may be used for the purpose of preventing or improving aging, or preventing or improving a disease inducing premature aging or a disease induced by aging.

The disease inducing premature aging may be, but is not limited to, Hutchinson-Gilford progeria syndrome. The term is as described above.

The disease induced by aging may be a disease induced by aging of vascular endothelial cells, a disease induced by aging of organs, a disease induced by aging of nerves, a degenerative brain disease, etc., and more specifically, it may be arteriosclerosis, liver failure, stroke, Alzheimer's disease, and Parkinson's disease, but is not limited thereto. The term is as described above.

The health functional food of the present disclosure may be prepared by a method commonly used in the art, and may be prepared by adding raw materials and ingredients that are conventionally added in the art. In addition, the health functional food may also be prepared in any formulation without limitation as long as the formulation is acceptable as a food. The health functional food composition of the present disclosure may be prepared in various formulations, and contains food as a raw material unlike general drugs, and thus has an advantage of having no side effects that may occur in long-term use of a drug, can be ordinarily ingested due to excellent portability and thus is very useful, and can be ingested as a supplement for enhancing effects of inhibiting, preventing, improving or delaying aging.

The health functional food is not particularly limited in other ingredients, except for the CRISPR/dCas9 complex, and may contain several herbal extracts, food supplement additives, or natural carbohydrates as additional ingredients, like in typical health functional foods. Additionally, the food supplement additives include typical food supplement additives in the art, for example, flavoring agents, savoring agents, coloring agents, fillers, stabilizers, etc.

Examples of the natural carbohydrates may include ordinary sugars, for example, monosaccharides, such as glucose, fructose, etc.; disaccharides, such as maltose, sucrose, etc.; and polysaccharides, such as dextrin, cyclodextrin, etc.; and sugar alcohols, such as xylitol, sorbitol, erythritol, etc. In addition to the above-described additives, natural flavoring agents (e.g., rebaudioside A, glycyrrhizin, etc.) and synthetic flavoring agents (saccharin, aspartame, etc.) may be advantageously used as the flavoring agents.

In addition to the above ingredients, the health functional food composition of the present disclosure may contain various nutrients, vitamins, water (electrolytes), flavoring agents, such as synthetic flavoring agents and natural flavoring agents, coloring agents, extenders (cheese, chocolate, etc.), pectic acid and salts thereof, alginic acid and salts thereof, organic acids, protective colloidal thickeners, pH adjusters, stabilizers, preservatives, glycerin, alcohols, carbonating agents used for carbonated drink, etc., and may also contain fruit flesh for preparing natural fruit juices, fruit juice drinks, and vegetable drinks. These ingredients may be used either alone or in combination. In addition, the health functional food may be in the form of any one of meat, sausage, bread, chocolate, candies, snacks, confectioneries, pizzas, ramen, gums, ice creams, soups, drinking water, teas, functional water, drinks, alcoholic drinks, and vitamin complexes.

Additionally, the health functional food of the present disclosure may further contain food additives, and the suitability thereof as a “food additive”, unless otherwise specified, is determined by the standards and criteria for the corresponding item in accordance with the General Rules and General Test Methods of the Food Additive Code approved by the Ministry of Food and Drug Safety.

The content of the composition that is added to food, in the process of preparing a health functional food, may be appropriately increased or reduced as needed.

Still another aspect of the present disclosure provides an injectable composition for preventing or treating an age-related disease, including any one selected from a composition including two or more proteins selected from GCLC, MAP1S, ENO1 and KNG1, or a polynucleotide encoding the proteins; or a composition which increases the expression of two or more proteins selected from GCLC, MAP1 S, ENO1 and KNG1.

The terms used in this aspect are as described above.

The composition of the present disclosure may further include any suitable excipient commonly used in injectable compositions, and such excipients may include, for example, preservatives, wetting agents, dispersing agents, suspending agents, buffers, stabilizers, or isotonic agents, etc., but are not limited thereto

Still another aspect of the present disclosure provides a composition for inhibiting cell aging, including any one selected from a composition including two or more proteins selected from GCLC, MAP1S, ENO1 and KNG1, or a polynucleotide encoding the proteins; or a composition which increases the expression of two or more proteins selected from GCLC, MAP1S, ENO1 and KNG1.

The terms used in this aspect are as described above

The inhibition of cell aging may be aging ax vivo, in vivo or in vitro.

The composition may be one that inhibits cell aging ax vivo, in vivo or in vitro, and for example, may be one that inhibits cell aging ex vivo or in vitro, but is not limited thereto.

### MODE FOR CARRYING OUT THE INVENTION

Hereinafter, the present disclosure will be described in more detail through exemplary embodiments to help understanding of the present disclosure. However, these exemplary embodiments may be modified in many different forms and the scope of the present disclosure should not be construed as being limited to the embodiments set forth below. The exemplary embodiments of the present disclosure are provided for the purpose of a full-depth explanation to those who have an average knowledge in the art.

### EXPERIMENTAL EXAMPLES

### <Experimental Example 1> Preparation of Plasma Treated with Oct4, Sox2, Klf4 and c-Myc(OSKM)

4F2A mice were fed with drinking water containing doxycycline for 4 weeks, and the expressions of Oct4, Sox2, Klf4, and c-Myc (OSKM) genes in vivo were induced. Thereafter, blood was isolated from 4F2A mice in which OSKM was induced and centrifuged at 3000 rpm for 30 minutes to isolate plasma (OSKM-treated plasma).

### <Experimental Example 2> Preparation of Mouse Models, and Injection of OSKM-Treated Plasma and Proteins Derived from OSKM-Treated Plasma

As a premature aging mouse model, mice overexpressing progerin (Progerin mouse, Jackson laboratory) were used. As an Alzheimer's mouse model, 5×FAD mice (Jackson laboratory) overexpressing five mutant genes related to amyloid-beta (Aβ) (three APP-related genes and two PSEN1-related genes) were used. A stroke mouse model was prepared by inducing middle cerebral artery occlusion (MCAO) using nylon thread. The MCAO induction can be performed with reference to the previous method (Im Byeong-cheol et al., The Journal of the Korea Contents Association, Vol. 19, No. 7, 2019). A Parkinson's disease mouse model was prepared by injecting 20 mg/kg of 1-methyl-4-phenyl-1, 2, 3, 6-tetrahydropyridine (MPTP) into the mouse abdomen. A liver failure mouse model was prepared by intraperitoneally administering a mixture of CCl4 (dimethylnitrosamine, thioacetamide): olive oil at a ratio of 1:3 (v/v) at 1 μL/g twice a week. Wild-type C57BL/6 male mice were obtained from the Korea Research Institute of Bioscience and Biotechnology.

All mice had free access to food and water and were maintained under a 12-h/12-h light/dark cycle at 23±1 °C. The mice were anesthetized with tribromoethanol (Avertin, 120 mg/kg) and injected with OSKM-treated plasma diluted in 0.9% saline (containing 0.9% w/v sodium chloride); or vectors expressing GCLC, MAP1S, ENO1, and KNG1 prepared in a polycistronic form into the tail vein. Specifically, lentivirus pelleted by ultracentrifugation was resuspended in 20 μl of PBS, and 5 μl of the virus was mixed with 300 μl of suspension and injected into the tail vein of mice. The titer of 5 μl of virus used here refers to the number of virus particles after monitoring GFP expression using FUW-GFP as a control and calculating the multiplicity of infection, before determining the virus titer (infection unit mL-1). The GCLC, MAP1S, ENO1 and KNG1 were expressed at the same ratio for the vectors expressing the GCLC, MAP1S, ENO1 and KNG1. After the injection, the mice were kept warm until they were fully recovered from anesthesia. The mice were biochemically and behaviorally analyzed two or four weeks after injection.

### <Experimental Example 3> Cell Cultivation

All cells were cultured in DMEM medium (Gibco) supplemented with 10% fetal bovine serum (FBS) and 1% penicillin/streptomycin (PIS) at 37 °C in 5% CO₂. Tail-tip fibroblasts (TTFs) were obtained from 1 to 2 mm sections of the tail-tip of the mice and washed with 70% ethanol for 30 seconds. The tissues were washed with fresh phosphate-buffered saline (PBS), minced with a sterile razor blade in a 10-cm tissue culture dish, and incubated with 0.25% trypsin (Gibco) containing ethylene-diamine-tetraacetic acid (EDTA) at 37 °C for 30 minutes.

### <Experimental Example 4> RNA Isolation and RT-qPCR

All RNAs were collected and purified using the eCube Tissue RNA Mini Kit (Philekorea) according to the manufacturer's instructions. RNA was then reverse transcribed into cDNA using AccuPower® CycleScript RT PreMix (Bioneer, #K-2044-B). RT-qPCR analysis was performed using AccuPower® PCR PreMix (Bioneer, #K-2016) with the primers listed in Table 1 below. All reactions were performed using a Rotor-Gene Q real-time PCR cycler (Qiagen).

TABLE 1

| SEQ | Name of | |
|---|---|---|
| ID NO: | Sequence | Sequence (5′→3′) |
| 9 | P21_F | CGGTGTCAGAGTCTAGGGGA |
| 10 | P21_R | ATCACCAGGATTGGACATGG |
| 11 | Atf3_F | CTCTGGCCGTTCTCTGGA |
| 12 | Atf3_R | GGTCGCACTGACTTCTGAGG |
| 13 | Btg2_F | GCGAGCAGAGACTCAAGGTT |
| 14 | Btg2_R | TAGCCAGAACCTTTGGATGG |
| 15 | Mmp13_F | TGATGAAACCTGGACAAGCA |
| 16 | Mmp13_R | GGTCCTTGGAGTGATCCAGA |
| 17 | Mmp12_F | AGTCCAGCCACCAACATTAC |
| 18 | Mmp12_R | GCTCCTGCCTCACATCATAC |
| 19 | IL-6_F | TGATGCACTTGCAGAAAACA |
| 20 | IL-6_R | ACCAGAGGAAATTTTCAATAGGC |
| 21 | IL-1a_F | GAAGAAGAGACGGCTGAGTTT |
| 22 | IL-1a_R | AACCAAGTGGTGCTGAGATAG |
| 23 | Cdkn1a_F | CGTGGACAGTGAGCAGTT |
| 24 | Cdkn1a_R | GACACCAGAGTGCAAGACA |
| 25 | Cdkn2a_F | CGTTCACGTAGCAGCTCTT |
| 26 | Cdkn2a_R | ATCGCACGATGTCTTGATGT |
| 27 | Cxcl1_F | CCGCTCGCTTCTCTGTG |
| 28 | Cxcl1_R | GACCATTCTTGAGTGTGGCTAT |
| 29 | Cxcl2_F | GTGAACTGCGCTGTCAATG |
| 30 | Cxcl2_R | GCCTTGCCTTTGTTCAGTATC |

### <Experimental Example 5> Western Blotting

Cells and tissues were homogenized by mixing RIPA buffer (50 mmol/L Tris containing 1% NP-40, 0.5% sodium deoxycholate (DOC), 0.1% SDS, and 150 mmol/L NaCl, pH 8.0, Sigma-Aldrich) with 1× protease inhibitor. Next, the samples were mixed with 5× loading buffer and boiled at 100 °C for 10 minutes. 10 μg of extracted protein was separated by SDS-PAGE on a 12% polyacrylamide gel and transferred to a nitrocellulose membrane (10600001, GE Healthcare). The protein concentrations were quantified using the Quick Start Bradford protein assay (Bio-Rad). The membranes were blocked in 5% bovine serum albumin (BSA) and incubated overnight at 4 °C with primary antibodies. The membranes were washed three times with PBS and then incubated with secondary antibodies for 2 hours at room temperature. Each protein band was visualized using an ECL kit (DG-WF200; Dogen) and quantified with the ImageJ (NIH) software. The antibodies used were anti-H3K9me3 (abcam, #ab8898), anti-H4K20me3 (abcam, #ab78517), α-SMA (Alpha Smooth Muscle Actin), and anti-β-actin (abfrontier, #LF-PA0207), and β-actin was used as a loading control.

### <Experimental Example 6> Test on Animal Behaviors

For behavioral analysis, an open field test was performed on mouse models. The mice were placed in a rectangular chamber (50 cm width, 50 cm length, 33 cm height), and the frequency with which the mice moved to the center of the rectangular chamber (25 cm width, 25 cm length) for 15 minutes was counted to measure the locomotion of the mice.

### <Experimental Example 7> Cell Staining

The mouse aorta was isolated and cells were fixed with 4% paraformaldehyde (PFA) to make paraffin blocks, and the aortic tissue was finely cut using a microtome. The thus-cut paraffin tissue samples were stained by H&E staining using hematoxylin and eosin. Additionally, VVG (Verhoeff Van Gieson), MT (Masson's Trichrome), and ORO (Oil Red 0) staining were performed using the cut paraffin tissue samples and kits (Verhoeff Van Gieson Elastic Stain Kit, Masson's Trichrome Stain Kit, Oil Red 0—Lipid Stain Kit) according to the manufacturers protocols.

### <Experimental Example 8> Immunofluorescence Staining

Cells were transformed with OSKM-treated plasma, or GCLC, Map1 s, ENO1, and KNG1-expressing vectors and incubated in 24-well plates for 24 hours. For immunofluorescence staining, the cells were washed with PBS and fixed with 4% paraformaldehyde for 10 minutes at room temperature. After washing twice with PBS, the cells were blocked with PBS containing 3% BSA and 0.1% Triton X-100 for 3 hours at room temperature. Primary antibodies were used at the dilution ratio recommended by the manufacturer. The cells were stained with primary antibodies at 4 °C for 24 hours, then incubated with secondary antibodies conjugated with Alexa488 or Alexa594 (Invitrogen), and stained with DAPI (4′,6-diamidino-2-phenylindole). Cell counts were calculated from 10 randomly selected fields in the plate at 200× magnification using a multipurpose confocal microscope (LSM-800, Zeiss). The antibodies used were anti-H3K9me3 (abcam, #ab8898), anti-H4K20me3 (abcam, #ab78517), α-SMA (Alpha Smooth Muscle Actin), and anti-β-actin (abfrontier, #LF-PA0207), and β-actin used as a loading control.

### <Experimental Example 9′ Liquid Chromatography-Tandem Mass Spectrometry (LC/MS) Analysis

Blood was obtained from the mice and centrifuged at 3000 rpm for 30 minutes to separate the supernatant serum. Then, qualitative and quantitative analysis of protein molecules in the separated serum was performed using LC/MS.

### <Experimental Example 10> Preparation of Vectors

The nucleotide sequences corresponding to the coding regions of Gclc, Eno1, Map1s, and Kng1 proteins were amplified by PCR from the mouse genomic DNA. The nucleotide sequences corresponding to each combination (GEMK, GEM, GEK, GMK, EMK, GE, GM, GK, EM, EK, MK) of Gclc, Eno1, Map1s, and Kng1 proteins were introduced into lentiviral vectors, and vectors for each combination were prepared through 2A sequences. The CMV promoter was used for the expression of each gene and combination genes. The thus-prepared vectors were prepared as lentiviruses.

### <Experimental Example 11> Preparation of Lentivirus Vectors

A lentiviral vector containing the mouse DNA (cDNA) complementary to FUW-TetO-mutant laminA/C (LMNA) and overexpressing progerin was prepared. HEK293T cells were cultured in DMEM medium containing 10% FBS and 1% P/S. The day before transfection, 2×10⁷ cells were seeded in a 15 cm culture dish containing medium. The following day, cells were co-transfected with lentiviral constructs psPAX2, pMD2.G and FUW-tetO-mutant LMNA via calcium phosphate co-precipitation. The medium was changed 24 hours after transfection, and viruses were harvested 72 hours after transfection. The supernatant containing lentivirus was collected, centrifuged to remove cell debris, and then filtered through a 0.45 μm filter. Lentivirus was pelleted by standard ultracentrifugation and resuspended in cold PBS. GFP expression was monitored using FUW-GFP as a control and the multiplicity of infection was calculated before determining the virus titer (infection units mL-1).

### <Experimental Example 12> Transfection

The day before transfection, the cells were seeded at 75 to 80% confluency to the plates. Three consecutive transfections were performed over 72 hours using Lipofectamine 3000 reagent (Thermo Fisher). Monolayers of cells in 6-well plates were transfected with constructs at a 5:2 or 6:2 ratio of Lipofectamine 3000 reagent to DNA and then incubated for 12 hours according to the manufacturer's protocol. The medium was changed 12 hours after transfection.

### <Experimental Example 13> Age-Related Beta-Galactosidase, SA-β-gal) Analysis

The cells were fixed with 4% paraformaldehyde for 5 minutes at room temperature, washed twice with PBS, and incubated overnight at 37 °C in a staining solution containing 40 mM citrate/Na phosphate buffer, 5 mM K4[Fe(CN)6] 3H₂O, 5 mM K3[Fe(CN)6], 150 mM sodium chloride, 2 mM magnesium chloride, and 1 mg/ml X-gal. The cells were washed twice with PBS and once with methanol, dried, and photographed using a bright-field microscope.

### <Experimental Example 14> Neurological Analysis

For the neurological analysis of the stroke mouse model, neurological severity score (NSS) and infarcted area volume were measured. For the NSS measurement, the Zea-longa score was determined by measuring whether the mice showed normal movement using the forelimbs in the cage. Additionally, the grab and hold ability test, which measured the ability to hold and maintain a bar with a height of 70 cm and a diameter of 0.5 cm with the forelimbs, and the circling behavior test, which measured the ability to maintain contact with the floor with the forelimbs, were performed. To measure the infarcted area volume, the mouse brain was isolated, and the cells were fixed with 4% PFA, and then sectioned into 0.45 μm thicknesses and stained Triphenyl tetrazolium chloride (TTC) and cresyl violet (CV).

### EXAMPLES

### <Example 1> Screening of Proteins in OSKM-treated Plasma and Improvement of Age-Related Markers in OSKM-treated Plasma and Proteins Derived from OSKM-treated Plasma

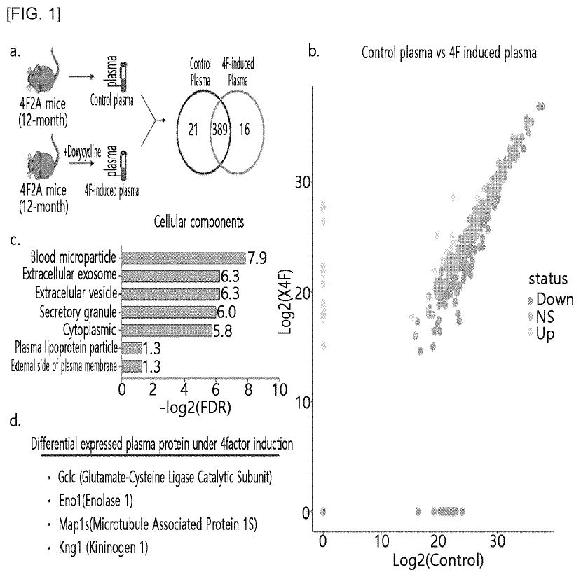

LC/MS was performed to analyze proteins in plasma derived from 4F2A mice in which OSKM was induced (OSKM-treated plasma) and compared with plasma derived from 4F2A mice in which OSKM was not induced (control group) (FIGS. 1a-c).

As a result, it was confirmed that 17 plasma proteins (GCLC, MAP1S, ENO1, KNG1, AFP, A2m, SAA2, YWHAG, PFKM, GDl2, ORM2, PCSK9, PSMD9, SNAP91, SLC17A9, PRG4, and SLC16A7) were specifically induced in OSKM-treated plasma compared to control plasma (FIG. 1d). Therefore, among the 17 proteins, 13 cancer-related proteins were excluded, and the remaining 4 proteins (GCLC, MAP1S, ENO1, and KNG1) were selected.

Next, aged mouse fibroblasts, which were transduced with mutant laminA/C (LMNA), were cultured for 5 days by treating with OSKM-treated plasma at a concentration of 10 μl/ml, or transformed intracellularly with vectors encoding the 4 proteins (GCLC, MAP1S, ENO1, and KNG1) among the 17 proteins to induce the expression of GCLC, MAP15, ENO1, and KNG1. After 5 days, the expression levels of age-related histone markers (H3K9me3 and H4K20me3) in each cell were analyzed by immunofluorescence staining.

As a result, when cells were treated with OSKM-treated plasma (FIGS. 2a-b) or the expression of GCLC, MAP1S, ENO1, and KNG1 derived from OSKM-treated plasma was induced (FIGS. 2c-d), the expression level of H3K9me3 was increased and the expression level of H4K20me3 was decreased, confirming that the age-related markers were improved.

Subsequently, OSKM-treated plasma was injected into 6-month-old (young) and 24-month-old C57BL/6 (old, naturally aged) mice, and then qRT-qPCR analysis was performed on stress response genes (p21, Btg2, and Atf), extracellular matrix-remodeling genes (MMP12, MMP13, and IL-6), senescence-associated secretory phenotype (SASP) genes (IL-1a, Cdkn1a, and Cdkn2a), and age-related genes (Cxcl1 and Cxcl2). As a result, the expression levels of age-related genes were significantly increased in fibroblasts derived from aged mice, but the expression levels of age-related genes were decreased in cells treated with OSKM-treated plasma (FIG. 5b).

Based on the above results, it was confirmed that when OSKM-treated plasma was treated or the expression of GCLC, MAP1S, ENO1, and KNG1 derived from OSKM-treated plasma was induced, the age-related markers were improved in cells, confirming that among the 17 proteins specifically expressed in OSKM-treated plasma, the above 4 proteins had aging inhibitory effects.

Wild-type mouse tail-tip fibroblasts were transformed with vectors [GCLC+ENO1(GE), GCLC+KNG1(GK), GCLC+MAP1S(GM), ENO1+MAP1S(EM), MAP1S+KNG1(MK), ENO1+MAP1S(EK), GCLC+ENO1+MAP1S(GEM), GCLC+ENO1+KNG1(GEK), GCLC+MAP1 S+KNG1 (GMK), ENO1+MAP1 S+KNG1(EMK), GCLC+MAP1 S+ENO1+KNG1(GEMK)] expressing various combinations of GCLC, MAP1S, ENO1, or KNG1, which were confirmed to have aging inhibitory effects among the 17 proteins specifically expressed in OSKM-treated plasma, and 3 days later, the number of age-related beta-galactosidase-positive cells and the intensity of cleaved-Caspase3 and gamma-H2AX, markers of cell aging that increase with the progression of aging, were measured.

As a result, the number of beta-galactosidase-positive cells was reduced when GCLC, MAP1S, ENO1, or KNG1 were treated alone, but when two or more thereof were treated in combination, the effect of reducing beta-galactosidase-positive cells was more superior. In particular, when combinations of 3 proteins [GCLC+ENO1+MAP1S (GEM), GCLC+ENO1+KNG1 (GEK), GCLC+MAP1S+KNG1 (GMK), and ENO1+MAP1S+KNG1 (EMK)] and a combination of 4 proteins [GCLC+MAP1S+ENO1+KNG1 (GEMK)] were treated, the effect of reducing beta-galactosidase-positive cells and the intensity of cleaved-Caspase3 and gamma-H2AX was the most superior, confirming that the level of reduction was similar to that of the control wild-type mouse fibroblasts (FIG. 3).

Based on the above results, when the expression of GCLC, MAP1S, ENO1, or KNG1 alone or in combination was induced, it was confirmed that age-related markers were improved in cells, thus confirming that the 4 proteins among the 17 proteins specifically expressed in OSKM-treated plasma had aging inhibitory effects.

In particular, when various combinations including two or more of the four proteins were expressed, as compared to when GCLC, MAP1S, ENO1, or KNG1 were expressed alone, the age-related markers were significantly improved in cells, confirming that the aging inhibitory effect was remarkable in the combination of the 4 proteins.

### <Example 2> Improvement of Age-Related Markers in Aged Mouse Model by Administration of OSKM-treated Plasma and Proteins Derived from OSKM-treated Plasma

It was determined whether the age-related phenotypes in vivo could be improved by administration of OSKM-treated plasma.

OSKM-treated plasma was injected into a 10-month-old premature aging mouse model to determine whether age-related markers were improved in the appearance, aorta, and organs.

After injection, the appearance of mice was significantly improved including a decrease in spinal curvature (FIG. 4c), weight loss was suppressed (FIG. 4a), and lifespan was increased (FIG. 4b), as compared to the control group.

Next, OSKM-treated plasma was injected into a premature aging mouse model, and after 4 weeks, stiffness and fibrosis symptoms in the aorta were analyzed using H&E, VVG, MT, and ORO staining. As a result, the stiffness and fibrosis symptoms in the aorta were improved in the mice after the injection as compared to the control group (FIGS. 4d-e).

In addition, the composition including GCLC, MAP1S, ENO1, and KNG1 was injected into naturally aged 24-month-old wild-type C57BL/6 mice, and after 4 weeks, stiffness and fibrosis symptoms in the aorta were analyzed using H&E staining. As a result, the stiffness and fibrosis symptoms in the aorta were improved in the mice after the injection compared to the control group (6-month-old mice) (FIGS. 6b-c).

Subsequently, OSKM-treated plasma was injected into a premature aging mouse model, and after 4 weeks, the aging symptoms in the liver, spleen, kidney, and skin were analyzed using H&E staining. As a result, the enlarged cells that increased with aging were reduced in the liver, and the size of the germinal center in the spleen was increased, in the mice after the injection as compared to the control group (5-month-old mice). Additionally, the number of glomeruli in the kidney was increased, and the epidermis thickness in the skin was increased, indicating an improvement in aging symptoms (FIGS. 4f-g).

In addition, after injecting the composition including GCLC, MAP1S, ENO1, and KNG1 into naturally aged 24-month-old wild-type C57BL/6 mice, the aging symptoms in the liver, spleen, and kidney were analyzed in the same manner. As a result, the symptoms of organ aging were improved in the mice after the injection as compared to the control group (6-month-old mice) (FIG. 6a).

Next, after injection of OSKM-treated plasma into 6-month-old (young), 16-month-old (mid-aged), and naturally aged 24-month-old (old) wild-type C57BL/6 mice, an open field test was performed. As a result, motor activity was increased in the mice after the injection as compared to the control group (FIG. 5a).

Subsequently, it was determined whether the administration of GCLC, MAP1S, ENO1, and KNG1 derived from OSKM-treated plasma could improve cardiac age-related phenotypes in vivo.

The composition including GCLC, MAP1S, ENO1, and KNG1 was injected into a 10-month-old premature aging mouse model and 24-month-old wild-type C57BL/6 mice, and after 4 weeks, the number of cardiac myofibroblasts in the aortic tissue was analyzed by α-SMA immunofluorescence staining. As a result, the number of α-SMA-positive cells was increased in the mice after the injection as compared to the control group, confirming an increase in the number of cardiac myofibroblasts (FIGS. 6d-e).

Based on the results, it was confirmed that the administration of OSKM-treated plasma and the composition including GCLC, MAP1S, ENO1 and KNG1 derived from OSKM-treated plasma improved age-related markers in the appearance, aorta and organs of the premature aging mouse model and aged mice.

### <Example 3> Improvement of Alzheimer's Disease-Related Markers in Alzheimer's Disease Mouse Model by Administration of Proteins Derived from OSKM-Treated Plasma

The composition including vectors expressing GCLC, MAP1S, ENO1, and KNG1 was injected into a 12-month-old Alzheimer's disease mouse model, and after 2 weeks, the levels of Alzheimer's disease-specific amyloid-beta (Aβ) plaques and Aβ42/40 ratios in brain tissues were analyzed.

As a result, the Aβ plaques and Aβ42/40 ratios were reduced in the mice after the injection as compared to the control group, confirming that the administration of the composition including GCLC, MAP1S, ENO1, and KNG1 improved the symptoms of Alzheimer's disease (FIG. 7).

### <Example 4> Improvement of Parkinson's Disease-Related Markers in Parkinson's Disease Mouse Model by Administration of Proteins Derived from OSKM-Treated Plasma

The composition including GCLC, MAP1S, ENO1, and KNG1 derived from OSKM-treated plasma was injected into a 12-month-old Parkinson's disease mouse model, and after 2 weeks, the mRNA expression levels of Map2, Synapsin, DAT, and TH, which are dopaminergic neuron-specific genes, were confirmed in the midbrain tissue.

As a result, the mRNA expression levels of Map2, Synapsin, DAT, and TH, which are dopaminergic neuron-specific genes, were increased in the mice after the injection as compared to the control group, confirming that administration of the composition including GCLC, MAP1S, ENO1, and KNG1 improved the symptoms of Parkinson's disease (FIG. 8).

### <Example 5> Improvement of Stroke-Related Markers in Stroke Mouse Model by Administration of Proteins Derived from OSKM-Treated Plasma

The composition including GCLC, MAP1S, ENO1, and KNG1 derived from OSKM-treated plasma was injected into a stroke mouse model, and after 2 weeks, the brain tissue was subjected to neurological analysis.

As a result, the infarct volume and neurological deficit score were reduced in the mice after the injection as compared to the control group, confirming that the administration of the composition including GCLC, MAP1S, ENO1, and KNG1 improved the symptoms of stroke (FIG. 9).

### <Example 6> Improvement of Liver Failure-Related Markers in Liver Failure Mouse Model by Administration of Proteins Derived from OSKM-Treated Plasma

The composition including GCLC, MAP1S, ENO1, and KNG1 derived from OSKM-treated plasma was injected into a liver failure mouse model, and after 2 weeks, plasma proteins were analyzed.

As a result, the levels of alanine transaminase (ATL) and aspartate transaminase (AST) were decreased and the level of plasma albumin (ALB) was increased in the mice after the injection as compared to the control group, confirming that administration of the composition including GCLC, MAP1S, ENO1, and KNG1 improved the symptoms of liver failure (FIG. 10).

From the results of the Examples above, OSKM-treated plasma and GCLC, MAP1S, ENO1 and KNG1 derived from OSKM-treated plasma improve age-related phenotypes in vitro and in vivo, and improve the symptoms of age-related diseases such as progeria, Alzheimer's disease, Parkinson's disease, stroke and liver failure, and thus can prevent, improve or treat age-related diseases.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the present disclosure is defined by the appended claims rather than by the description preceding them, and therefore all changes and modifications that fall within metes and bounds of the claims, or equivalents of such metes and bounds are therefore intended to be embraced by the claims.

1. A method for preventing or treating an age-related disease, including administering a composition, which includes two or more proteins selected from GCLC, MAP1S, ENO1 and KNG1, or a polynucleotide encoding the proteins; or a composition which increases the expression of two or more proteins selected from GCLC, MAP1S, ENO1 and KNG1, as an active ingredient.

2. (canceled)

3. The method of claim 1, wherein the GCLC protein comprises an amino acid sequence of SEQ ID NO: 1.

4. The method of claim 1, wherein the MAP1S protein comprises an amino acid sequence of SEQ ID NO: 3.

5. The method of claim 1, wherein the ENO1 protein comprises an amino acid sequence of SEQ ID NO: 5.

6. The method of claim 1, wherein the KNG1 protein comprises an amino acid sequence of SEQ ID NO: 7.

7. The method of claim 1, wherein the composition, which increases the expression of two or more proteins selected from GCLC, MAP1S, ENO1 and KNG1, is a vector containing two or more of the following: a polynucleotide encoding the GCLC protein; a polynucleotide encoding the MAP1S protein; a polynucleotide encoding the ENO1 protein; and a polynucleotide encoding the KNG1 protein.

8. The method of claim 1, wherein the composition has a cell aging inhibitory effect.

9. The method of claim 8, wherein the cell aging inhibitory effect is any one or more effects selected from the following:
i) reduction in the expression level of age-related genes;
ii) reduction in the level of DNA double-strand breaks; and
iii) reduction in the number of beta-galactosidase positive cells.

10. The method of claim 1, wherein the pharmaceutical composition has an effect of improving an age-related disease.

11. The method of claim 10, wherein the effect of improving an age-related disease is any one or more effects selected from the following:
i) reduction of spinal curvature;
ii) suppression of weight loss;
iii) increase in life span;
iv) increase in motor activity; and
v) improvement of aging of organs and skin.

12. The method of claim 1, wherein the age-related disease is any one or more selected from a disease inducing premature aging or a disease induced by aging.

13. The method of claim 12, wherein the disease inducing premature aging is any one or more selected from the group consisting of Hutchinson Guilford progeria syndrome (HGPS), progeria, premature aging associated with HIV infection, muscular dystrophy, Charcot-Marie-Tooth disease, Werner syndrome, insulin-resistant type II diabetes, osteoporosis, skin aging, and restrictive dermopathy.

14. The method of claim 12, wherein the disease induced by aging is any one or more selected from the group consisting of arteriosclerosis, liver failure, stroke, degenerative brain disease, Alzheimer's disease, Parkinson's disease, hypertension, cognitive dysfunction, sarcopenia, dry eye, macular degeneration, hyperopia, myopia, cataracts, tinnitus, hearing loss, indigestion, diarrhea, autoimmune disease, pneumonia, influenza, tetanus, infective endocarditis, cancer, overactive bladder, urinary incontinence, benign prostatic hyperplasia, lower urinary tract symptoms, glomerulonephritis, chronic renal failure, osteoporosis, arthritis, diabetes, renal failure, chronic obstructive pulmonary disease, and pulmonary fibrosis.

15. (canceled)

16. A method for inhibiting cell aging, including administering any one selected from: a composition comprising two or more proteins selected from GCLC, MAP1S, ENO1, and KNG1 or a polynucleotide encoding the same; or a composition which increases the expression of two or more proteins selected from GCLC, MAP1S, ENO1, and KNG1.

17. The method of claim 16, wherein the composition inhibits cell aging in vitro or ex vivo.


SEQUENCE LISTING (30 sequences)

SEQ ID NO: 1 - protein, 637 aa, Mus musculus
MGLLSQGSPLSWEETQRHADHVRRHGILQFLHIYHAVKDRHKDVLKWGDEVEYMLVSFDHENRKVQLLLNGGDVLETLQEKGERTNPNHPTLWRPEYGSYMIEGTPGQPYGGTMSEFNTVEANMRKRRKEATSVLGEHQALCTITSFPRLGCPGFTLPEHRPNPEEGGASKSLFFPDEAINKHPRFGTLTRNIRHRRGEKVVINVPIFKDKNTPSPFVETFPEDAEASKASQPDHIYMDAMGFGMGNCCLQVTFQACSISEARYLYDQLATICPIVMALSAASPFYRGYVSDIDCRWGVISASVDDRTREERGLEPLKNNRFRISKSRYDSIDSYLSKCGEKYNDIDLTIDKEIYEQLLEEGIDHLLAQHVAHLFIRDPLTLFEEKIHLDDANESDHFENIQSTNWQTMRFKPPPPNSDIGWRVEFRPMEVQLTDFENSAYVVFVVLLTRVILSYKLDFLIPLSKVDENMKVAQKRDAVLQGMFYFRKDICKGGNAVVDGCSKAQSSSEPAAEEYTLMSIDTIINGKEGVFPGLIPILNSYLENMEVDVDTRCSILNYLKLIKKRASGELMTVARWMREFIANHPDYKQDSVITDEINYSLIWKCNQIADELCECPELLGSGFRKAKYSGGKSDPSA

SEQ ID NO: 2 - DNA, 1914 bp, Mus musculus
  source 1..1914: mol_type = genomic DNA
atggggctgctgtcccaaggctcgccactgagctgggaagagacccagcgccacgccgaccacgtgcggaggcacggcatcctccagttcctgcacatctaccacgcagtcaaggaccggcacaaggacgtgctcaagtggggtgacgaggtggagtacatgttggtgtccttcgatcatgaaaataggaaagtccagttgttactgaatggaggcgatgttcttgagactctgcaggagaagggggagaggacaaaccccaaccatccgaccctctggagaccagagtatgggagttacatgatcgaaggaacgcctggccagccctacggaggaacgatgtctgagttcaacactgtggaggccaatatgaggaaacgccggaaggaggctacttctgtactaggagagcatcaggctctctgcaccatcacttcattccccagactaggctgtcctggattcacactgccagaacacagacccaacccagaggaaggaggcgcatcaaagtccctcttctttccagatgaagccataaacaagcacccccgcttcggtactctaacaagaaacatccggcatcggagaggagaaaaggttgtcatcaatgtgccaatattcaaggacaagaatacaccatctccatttgtagaaacatttccagaagatgcggaggcatcaaaggcttctcagccagaccatatctacatggatgccatgggattcgggatgggcaactgctgtctccaggtgacattccaagcctgcagcatatctgaggcaagatacctttatgatcagctggccactatctgcccaattgttatggctttgagtgctgcatctccattttaccgaggctacgtgtcagacattgattgtcgctggggagtgatttctgcctctgtagatgatagaacacgggaggagagaggcctggagcctctgaagaacaatcgctttaggatcagtaagtctcggtatgactcaatagatagctacctgtctaagtgtggggagaagtacaatgacatcgacctgaccatcgataaggagatctacgagcagctcctggaggaaggcatcgatcatcttctggcacagcacgttgctcatctctttattagagacccactcaccctttttgaagagaaaatacacctggatgatgccaacgagtctgaccattttgagaatattcagtctacaaactggcagacaatgaggtttaagcctcctcctccaaactcagatattggatggagagtagagttccgaccaatggaggtgcagttaacagactttgagaactctgcctatgtggtattcgtggtactgctcactagggtgatcctctcatacaaactagacttcctcattccgctgtccaaggttgacgagaacatgaaagtggcccagaagcgagatgctgtcttgcaggggatgttttatttcaggaaagacatctgcaaaggcggcaacgctgtggtggacgggtgcagcaaggcccagagcagctctgagccagctgcagaggagtacaccctgatgagcatagacaccatcatcaatgggaaggaaggggtatttcctggactcatccccattctgaactcctatctggagaacatggaagttgatgtggacacccgatgcagtattctgaactacctaaagctaattaagaagagagcatctggagaactaatgactgttgccaggtggatgagagagttcattgcaaaccatcctgactacaagcaagacagtgtaataactgatgaaatcaactatagcctcatttggaagtgcaaccaaattgcagatgagctgtgtgaatgtccagagttacttggatcaggatttagaaaagcgaagtacagtggaggtaaaagcgacccttcagcctag

SEQ ID NO: 3 - protein, 973 aa, Mus musculus
MAAVMAAPEAVEAPSSLLLLVVGGECGCPGLLAYVMEELERGVRSWEDVDPAVCSLDEQLKAFVSRHSATFSSIVKGQRSLHHRGETLETLVLLNPSDKSLCDELRNLLMDPAPHKLLVLAGPCLEETGELLLQTGGFSAHHFLQVLGDKEVQDALASAPAAPALTVSCPTFGDWALLGPVPGLQLRLNPPAQLPASEGLRAFLEYVAESLEPPSPFELLEPPAAGGFLRLARPCCYVFPGGLGDAAFFAVNGFTVLVNGGSNPKSSFWKLVRHLDRVDAVLVTHAGADSLPGLNSLLRRKLAEREAAAGPQGQHEERLRRLLSPALGVVFLNAREAASRLRGGEDEAVCARSLLRSLGIAPLPLQRGPQPSCPTVLFEKLGVGRLELFVLHPPPGDPAAPACALLVWQPAAPGDKVVRVLFPGRTPPARLLDGLQRLQHLPCLRRPVVTTHDLEAPSRANSQDSLASRDSARKEPVRGTVGSIANRSTVRREPALATRDQKKDTRSGPTQPTARDTRRSGPGVVNTKPRVSQNGPRAPVLAAPLTAPVAECPGEAENILESERPPAPSPTLSPAQSPPPTAPGNSPERLSLSPLRPEPAPDASPSATTPTLTTPSLPAELGSPHSTEVDESLSVSFEQVLPAGDPGLSLPLRLARRSTSPHDVDLCLVSPCEFSHRKPPPPASPGSSDSSARSQERPPETPPTSVSESLPTLSDSDPVPVADSDDDAGSESAARDPPPTPRVPPPLPDVPGICMVDPEALPPRARQPLNTTNPSRSRKAPARPSSASATPRAATVAAKTKGPAGDRNRPLSARSEPADRPGRVPLTRKPSVPKTVPKMASATRLSSGPSGRPAPLAAGSPVYLDLAYLPGGGAGHLDQNFFLRVRALCYVISGQGQRQEEGLRAVLDALLAGKRQWDLDLQVTLIPTFDSAVMHRWYEETHAQHQALGIRVLGSGSLVSMQDEAFPACKVEF

SEQ ID NO: 4 - DNA, 2922 bp, Mus musculus
  source 1..2922: mol_type = genomic DNA
atggcggcagtgatggcggcgccggaggctgtggaggcgcccagctccctactgttgctggtggtgggtggcgagtgtggctgccccgggctgctggcctacgtcatggaggagttggaacgaggtgttcgctcctgggaggatgtggaccccgctgtctgcagcctggatgagcagctcaaggccttcgtgtcccggcattcagccaccttctccagcattgtgaaagggcagcggagcctgcaccaccgtggagagaccttggagacgctggtcctgctgaacccctcagacaagtccctgtgtgatgagctccggaacctcctgatggaccccgcccctcacaagctgctggtcctggccgggccctgcctggaggagactggggaactgctgttgcagactggggggttctcggcccaccacttcctgcaggtgctgggggacaaagaggtccaggatgcactggcctcagcaccagctgccccagccctcactgtgtcctgtccaacctttggggactgggcactgttgggccctgtgcccgggctgcagctgcgactaaatccgcccgcgcagctgccggcctcagagggccttcgtgccttcctggagtatgtggctgagtcgctggagccgccatcgcccttcgagttgttggagccaccggctgcgggcggctttctgcgcttagcaaggccctgttgctatgtgttcccgggtggcttgggagacgccgccttcttcgctgtcaacggctttacagtgctggtgaatggtggctccaaccccaagtcgagcttctggaagctggtgcgccacctggatcgcgtggacgctgtccttgtgacccacgcgggtgctgacagcttgccgggcctcaacagcctgctgcgccgcaagctggcagagcgcgaagcagccgcaggaccacagggacagcatgaggagcggctacgtcgcctgctgtcgcctgcgctgggtgtggtgttcctgaatgctcgtgaggctgcttctcggttgcgtgggggcgaagacgaggctgtgtgtgctcggagcctgctgcggagcctgggcattgcgcctctgccactgcagcgtggcccacagccctcttgccccactgtccttttcgagaagctgggcgtaggccgcctagagctttttgtgctgcaccctcctcctggggaccctgcagcgcctgcctgtgctctgctcgtgtggcagccagcggcgcctggcgacaaggttgtgcgtgtactatttcctggccgcacgccgcccgcgcgcctgctggatgggctacagcgcctgcagcacctgccgtgcctgcgcaggccagtagttaccacgcatgacctggaagcgccctccagggccaacagccaagacagtctggcctcgcgggacagtgcacgcaaggagccagtccgtggcactgttggcagcattgccaacaggagtacagtgcgaagggagcctgccctggccactcgtgaccagaagaaggacacaagatctgggcccactcagcctactgcccgagacacacgccgctccgggcctggtgtggtcaacacaaagcctcgtgtatcacagaatgggcctcgtgccccagttctggcagcgccactcacagctcctgtggctgagtgccctggagaagcggagaacatcttggagtctgagcggccacctgccccatccccaactctgtccccagctcagtccccaccacctacagcacctggtaatagccctgagcgcctgtcactcagccctctgcgcccagagcctgcccctgatgcgtcgccctcagccacgacgcccacgctgaccacgccctctttgcctgcggagctgggctcaccccactcaactgaggtggacgagtcgctctctgtgtctttcgagcaagtcctgccagcaggtgacccagggcttagcctgcccctgcgcctcgcgcgccgctccacgtctcctcatgatgtggacctgtgcctcgtgtcaccctgtgagttctctcatcgcaagccaccccctcctgcctccccaggcagctcggacagcagtgctcggtcacaggagcggccacctgagactccacccacgtctgtgagcgagtcactgcccacactatctgactctgatcccgtgcccgtggcggattctgatgatgacgcaggcagcgagagcgcggccagggacccgccgcctacacctcgtgtgccaccaccactgccagatgtaccaggaatctgcatggtggacccggaggcgctaccgcctcgtgcccggcagcccctcaacaccaccaatcccagccgtagccgcaaggcccctgcaaggcctagctctgcctctgccacccccagagctgcaactgtagctgccaagacaaagggccccgcgggggaccggaaccggccactcagcgcccgcagtgagcctgcagacaggccaggccgtgtgccccttaccaggaagccctcagtccctaagaccgttcccaagatggcctctgccacaaggctctcctccggacccagtggccgccctgcaccgcttgctgctggctcccctgtatacctggacctggcctacctgcctggtggtggtgccggtcacctggaccagaacttcttcctgcgtgtccgtgcactctgctatgtcatcagtgggcaaggccagcgccaggaagagggcctgcgagctgtgctggatgcactgctggctggcaagcggcagtgggaccttgacttgcaggtcacactgatccccaccttcgactctgcggtgatgcacaggtggtatgaggagacgcatgcgcagcaccaggcactgggcatcagggtgctgggcagtggcagcctggtgtccatgcaggacgaggccttccccgcctgcaaggtggagttctag

SEQ ID NO: 5 - protein, 434 aa, Mus musculus
MSILRIHAREIFDSRGNPTVEVDLYTAKGLFRAAVPSGASTGIYEALELRDNDKTRFMGKGVSRPVKYVNEFLAPALCTQKVNVVEQEKIDKLMIEMDGTENKSKFGANAILGVSLAVCKAGAVEKGVPLYRHIADLAGNPEVILPVPAFNVINGGSHAGNKLAMQEFMILPVGASSFREAMRIGAEVYHNLKNVIKEKYGKDATNVGDEGGFAPNILENKEALELLKTAIAKAGYTDQVVIGMDVAASEFYRSGKYDLDFKSPDDPSRYITPDQLADLYKSFVQNYPVVSIEDPFDQDDWGAWQKFTASAGIQVVGDDLTVTNPKRIAKAASEKSCNCLLLKVNQIGSVTESLQACKLAQSNGWGVMVSHRSGETEDTFIADLVVGLCTGQIKTGAPCRSERLAKYNQILRIEEELGSKAKFAGRSFRNPLAK

SEQ ID NO: 6 - DNA, 1305 bp, Mus musculus
  source 1..1305: mol_type = genomic DNA
atgtctattctcaggatccacgccagagagatctttgactcccgtgggaatcccactgttgaggtcgatctgtacaccgcaaaaggtctcttccgagctgcggtgcccagcggtgcgtccactggcatctacgaggccctagaactccgagacaatgataagacccgcttcatggggaagggcgtctccagacccgttaagtatgttaatgagttcctggcaccagccttgtgcactcagaaagtgaatgttgtggagcaagagaagattgacaagctgatgatcgagatggacggcacagagaataaatctaaatttggtgcaaatgccatcctgggagtgtccctggctgtctgcaaagctggtgccgtggaaaagggggtgcccctttaccgccacattgctgacttggccggcaaccctgaagtcatcctgcctgtcccggctttcaatgtgatcaacggtggttctcatgctggcaacaagctggccatgcaagagttcatgatcctgcctgtgggggcatccagcttccgggaagccatgcgcattggagcagaggtttaccacaacctgaagaacgtgatcaaggagaagtacgggaaggacgccaccaatgtgggtgatgagggtggattcgcacctaacatcctggagaacaaagaagcactggagctgctcaagactgcaatcgcaaaggccggctacactgaccaggttgtcattggcatggatgtggctgcctccgagttctacaggtctggcaagtatgacctggacttcaagtctccggatgaccccagcaggtacatcactcccgaccagctggctgatctgtacaagtccttcgtccagaactacccagtggtgtccatcgaagatccctttgaccaggacgactggggcgcctggcagaagttcacggctagtgcgggcatccaggtggtgggcgatgacctcacagtgaccaaccctaagcggattgccaaggctgcgagcgagaagtcctgcaactgcctcttgctcaaagtgaaccagatcggctctgtgaccgaatccctgcaggcgtgtaagctggcccaatccaatggctggggcgtcatggtgtcccaccgatctggggaaactgaggacactttcatcgcagacctggtggtggggctctgcactgggcagatcaagactggtgccccttgccgatccgagcgcctggccaagtacaatcagatcctcagaattgaggaagagctgggcagcaaagccaagtttgctggcaggtccttcaggaaccccctggccaaataa

SEQ ID NO: 7 - protein, 661 aa, Mus musculus
MKLITTLLLCSGLLLTLTQGEEAQEIDCNDEAVFQAVDFSLKQFNPGVKSGNQYMLHRVIEGTKTDGSPTFYSFKYLIKEGNCSAQSGLAWQDCDFKDAEEAATGECTATVGKRENEFFIVTQTCKIAPSKAPILKAYFPCIGCVHAISTDSPDLEPVLKHSIEHFNNNTDHSHLFTLRKVKSAHRQVVAGLNFDITYTIVQTNCSKERFPSLHGDCVALPNGDDGECRGNLFMDINNKIANFSQSCTLYSGDDLVEALPKPCPGCPRDIPVDSPELKEVLGHSIAQLNAENDHPFYYKIDTVKKATSQVVAGTKYVIEFIARETKCSKESNTELAEDCEIKHLGQSLDCNANVYMRPWENKVVPTVKCQALDMTEMARRPPGFSPFRSVTVQETKEGRTVSPPYIAREQEERDAETEQGPTHGHGWLHEKQIKANKNHRGHKHGHDHGHWSPRRHGLGHGHQKPHGLGHGHQLKLDYLRHQREDGDDHTHTVGHGHGHGHGHGHGHGHGHGHGHGHGHGHGHGKHTNKDKNSVKQTTQRTESLASSSEYSTTSTQMQGRTEGPTLTPPRAQPTVTSSGFQDSDFIEDVVATTPPYDTGAHDDLIPDIHVQPDSLSFKLISDFPEATSPKCPGRPWKPASWKDPNTETTEFSDFDLLDALS

SEQ ID NO: 8 - DNA, 1986 bp, Mus musculus
  source 1..1986: mol_type = genomic DNA
atgaagctcattactacactgctcctctgctccggactcctgctgactttaacacagggagaagaagcgcaggaaattgactgcaatgatgaggctgtatttcaggctgtggatttctctctgaagcagtttaaccctggggtaaaaagtggcaaccagtatatgttgcaccgagtgatcgagggcactaaaacggatggctctccaaccttttactccttcaagtatctaatcaaggagggcaactgctctgctcagagtggcctcgcatggcaggactgtgacttcaaggacgctgaggaagccgccactggagaatgcacagcaactgtggggaaaagagaaaatgaattcttcatagtcacccagacctgcaagattgctccaagtaaggcccccatactgaaagcctatttcccctgtattggttgtgtgcatgccatatcgacagatagtccagacctggagcctgttctgaaacactccatcgaacatttcaacaacaacacagatcacagccacctctttactctcagaaaagtaaaaagtgcccacagacaggtggtggctggcctgaattttgacattacctacacaattgtgcaaacaaattgttcaaaggagcgttttccttccctccatggagactgcgtggcccttcccaatggtgatgatggtgaatgtagaggaaatctcttcatggatattaataacaaaattgccaacttctcacagagctgtaccctttattcaggagatgatttggtagaagcgcttcccaagccttgccctggctgccccagggacatacctgtagacagcccagagctgaaggaggtgcttggtcattccattgcacagctaaatgcagagaatgaccatcctttctattacaagattgacaccgtgaaaaaagcaacatcacaggtggtagcaggaactaaatatgttattgagttcatagccagagaaaccaaatgctccaaggaaagtaacacagagctggcagaagattgtgagatcaagcaccttggacaaagtctcgactgcaatgctaacgtgtacatgagaccttgggagaacaaagtcgtcccgactgtgaaatgccaagcattagatatgactgaaatggcaagaaggcctccaggtttttctcctttccggagtgtcacagtacaagaaacaaaagaaggaagaactgtaagtccaccctacattgccagggagcaagaagagagggatgcagaaactgaacaaggacccacacatgggcatggctggttgcatgaaaagcaaatcaaggctaataagaatcaccgtggtcataagcatgggcatgaccatggccattggtccccaaggaggcatggccttggtcatggacaccagaaaccacatggccttggtcatggacatcaacttaaacttgattatcttagacaccaaagggaagatggtgatgaccatacacacacagtgggacatggtcatggtcacggacatggtcatggtcatggacatggacatggtcatggtcacggtcatggtcatggtcatggacatggtcatggtaagcatacaaataaagacaaaaacagtgtaaagcagactacccagagaacagagtctttggcaagctcttctgaatacagtactacatctacacagatgcaggggaggacagaagggcccaccttgacccctccccgagcccagccaactgttacctcttctggttttcaggactcagatttcattgaagatgtggtagctaccaccccaccatatgacactggggcccatgatgatttgatccctgatatccatgtacagccagatagcctttcatttaagctgatatctgattttccagaagcaacttcccccaaatgtcctgggcgcccatggaagccagctagctggaaggatccaaacacagaaacaacagaattttctgattttgatctccttgatgctctttcttaa

SEQ ID NO: 9 - DNA, 20 bp, synthetic construct
  source 1..20: mol_type = other DNA
cggtgtcagagtctagggga

SEQ ID NO: 10 - DNA, 20 bp, synthetic construct
  source 1..20: mol_type = other DNA
atcaccaggattggacatgg

SEQ ID NO: 11 - DNA, 18 bp, synthetic construct
  source 1..18: mol_type = other DNA
ctctggccgttctctgga

SEQ ID NO: 12 - DNA, 20 bp, synthetic construct
  source 1..20: mol_type = other DNA
ggtcgcactgacttctgagg

SEQ ID NO: 13 - DNA, 20 bp, synthetic construct
  source 1..20: mol_type = other DNA
gcgagcagagactcaaggtt

SEQ ID NO: 14 - DNA, 20 bp, synthetic construct
  source 1..20: mol_type = other DNA
tagccagaacctttggatgg

SEQ ID NO: 15 - DNA, 20 bp, synthetic construct
  source 1..20: mol_type = other DNA
tgatgaaacctggacaagca

SEQ ID NO: 16 - DNA, 20 bp, synthetic construct
  source 1..20: mol_type = other DNA
ggtccttggagtgatccaga

SEQ ID NO: 17 - DNA, 20 bp, synthetic construct
  source 1..20: mol_type = other DNA
agtccagccaccaacattac

SEQ ID NO: 18 - DNA, 20 bp, synthetic construct
  source 1..20: mol_type = other DNA
gctcctgcctcacatcatac

SEQ ID NO: 19 - DNA, 20 bp, synthetic construct
  source 1..20: mol_type = other DNA
tgatgcacttgcagaaaaca

SEQ ID NO: 20 - DNA, 23 bp, synthetic construct
  source 1..23: mol_type = other DNA
accagaggaaattttcaataggc

SEQ ID NO: 21 - DNA, 21 bp, synthetic construct
  source 1..21: mol_type = other DNA
gaagaagagacggctgagttt

SEQ ID NO: 22 - DNA, 21 bp, synthetic construct
  source 1..21: mol_type = other DNA
aaccaagtggtgctgagatag

SEQ ID NO: 23 - DNA, 18 bp, synthetic construct
  source 1..18: mol_type = other DNA
cgtggacagtgagcagtt

SEQ ID NO: 24 - DNA, 19 bp, synthetic construct
  source 1..19: mol_type = other DNA
gacaccagagtgcaagaca

SEQ ID NO: 25 - DNA, 19 bp, synthetic construct
  source 1..19: mol_type = other DNA
cgttcacgtagcagctctt

SEQ ID NO: 26 - DNA, 20 bp, synthetic construct
  source 1..20: mol_type = other DNA
atcgcacgatgtcttgatgt

SEQ ID NO: 27 - DNA, 17 bp, synthetic construct
  source 1..17: mol_type = other DNA
ccgctcgcttctctgtg

SEQ ID NO: 28 - DNA, 22 bp, synthetic construct
  source 1..22: mol_type = other DNA
gaccattcttgagtgtggctat

SEQ ID NO: 29 - DNA, 19 bp, synthetic construct
  source 1..19: mol_type = other DNA
gtgaactgcgctgtcaatg

SEQ ID NO: 30 - DNA, 21 bp, synthetic construct
  source 1..21: mol_type = other DNA
gccttgcctttgttcagtatc